(19) United States
(12) Patent Application Publication
(10) Pub. No.: US 2026/0290543 A1
(43) Pub. Date: Sep. 24, 2026

(54) SYSTEM

(71) Applicant: SoftBank Group Corp., Tokyo (JP)

(72) Inventor: Sui HOTATE, Tokyo (JP)

(73) Assignee: SoftBank Group Corp., Tokyo (JP)

(21) Appl. No.: 19/561,563

(22) Filed: Mar. 10, 2026

(30) Foreign Application Priority Data
Mar. 19, 2025 (JP) 2025-044472

(51) Int. Cl. G16H 20/60; G06N 3/0475

(52) U.S. Cl. CPC G16H 20/60; G06N 3/0475

(57) ABSTRACT

A system includes a processor that is configured to provide an interface for receiving input information from a user, analyze the received input information and generate a prompt for instructing a generative AI model to generate a recipe that matches a meal preference of the user, and input the generated prompt into the generative AI model to cause the generative AI model to generate a recipe based on the meal preference of the user.

### CROSS-REFERENCE TO RELATED APPLICATION

This application is based on and claims priority under 35 USC 119 from Japanese Patent Application No. 2025-044472 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Traditional recipe recommendation and generation systems have difficulty accurately reflecting an individual user's detailed meal preferences and contextual needs. Conventional approaches often rely on static rule-based engines or coarse user profiles, which cannot flexibly handle natural language expressions of preferences, restrictions, or nuanced feedback. As a result, users frequently receive recipes that do not sufficiently match their tastes, dietary constraints, or evolving preferences. Furthermore, existing systems typically do not effectively utilize past user feedback data to refine subsequent recipe generation in a personalized manner. There is therefore a need for a system that can receive natural language input from a user, interpret such input appropriately, generate prompts suitable for a generative AI model, and cause the generative AI model to generate recipes that closely match the user's meal preferences, while also learning from the user's past feedback data.

### SUMMARY

In order to solve the above-mentioned problems, the present invention provides a system comprising a processor, wherein the processor is configured to provide an interface for receiving input information from a user, analyze the received input information, and generate a prompt for instructing a generative AI model to generate a recipe that matches a meal preference of the user. The processor is further configured to input the generated prompt into the generative AI model to cause the generative AI model to generate a recipe based on the meal preference of the user. In some embodiments, the processor is configured to analyze the prompt by using a natural language processing technique and generate the recipe that matches the meal preference of the user. In some embodiments, the processor is configured to refer to past feedback data of the user and generate a prompt that reflects a preference of the user. By combining natural language analysis of user input, prompt generation for a generative AI model, and utilization of past feedback data, the system can dynamically provide recipes that are more accurately tailored to the individual user's preferences and constraints.

The term “system” refers to an arrangement of hardware and software components, including at least one processor and associated memory and interfaces, that cooperate to execute the processing described in the claims.

The term “processor” refers to one or more hardware processing units, such as a central processing unit (CPU), a graphics processing unit (GPU), a digital signal processor (DSP), or any combination thereof, capable of executing instructions to perform the functions recited in the claims.

The term “interface” refers to a hardware and/or software component that enables the exchange of information between the user and the system, including, for example, graphical user interfaces, command-line interfaces, application programming interfaces (APIs), touch screens, keyboards, microphones, and network communication interfaces.

The term “input information” refers to data provided by the user to the system via the interface, including but not limited to natural language text, spoken language converted to text, structured preference settings, constraints, and any other information that describes the user's meal preferences or requirements.

The term “user” refers to a person or entity that interacts with the system by providing input information and receiving recipes generated by the system.

The term “prompt” refers to a data structure or text sequence generated by the processor, designed to be provided as input to a generative AI model, and including instructions, constraints, and contextual information for causing the generative AI model to generate a recipe.

The term “generative AI model” refers to a machine learning model, such as a neural network-based language model or multimodal model, that is trained to generate new content, including recipes expressed in natural language, in response to a given prompt.

The term “recipe” refers to a structured or unstructured representation of cooking-related information, including, for example, a list of ingredients, quantities, preparation steps, cooking methods, and serving suggestions that describe how to prepare a meal.

The term “meal preference” refers to a set of characteristics, constraints, and tendencies associated with the user's desired meals, including tastes, preferred ingredients, disliked ingredients, allergies, nutritional requirements, cooking methods, cuisines, portion sizes, and any other relevant factors.

The term “natural language processing technique” refers to any algorithmic method or model that processes and analyzes human language input or prompts, including tokenization, syntactic parsing, semantic analysis, intent recognition, entity extraction, and language modeling.

The term “past feedback data” refers to historical information provided by the user to the system regarding previously generated or consumed recipes, including numerical ratings, textual comments, explicit acceptances or rejections, and other evaluation data that reflect the user's satisfaction or dissatisfaction.

The term “preference of the user” refers to a representation, learned or explicitly specified, of the user's likes, dislikes, constraints, and priorities regarding meals, which is derived from input information, past feedback data, or both, and is used by the processor to generate or adjust prompts.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

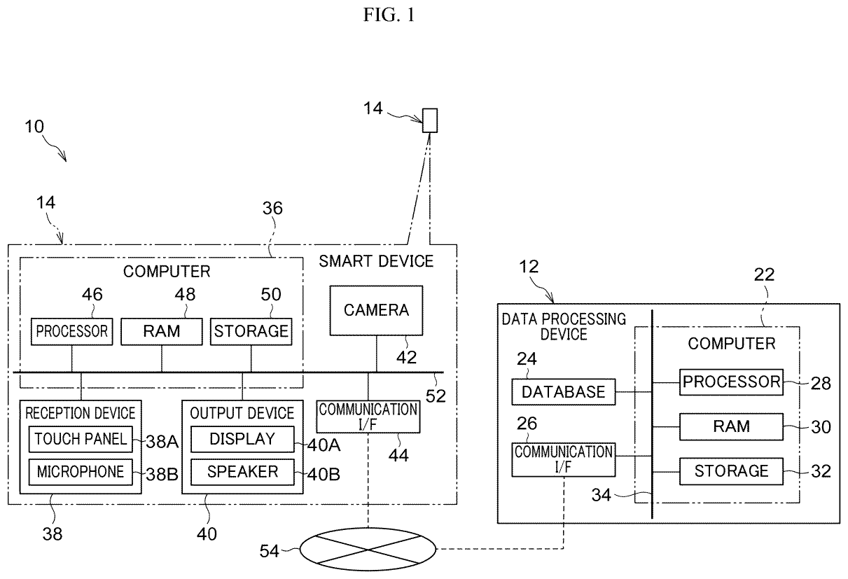

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

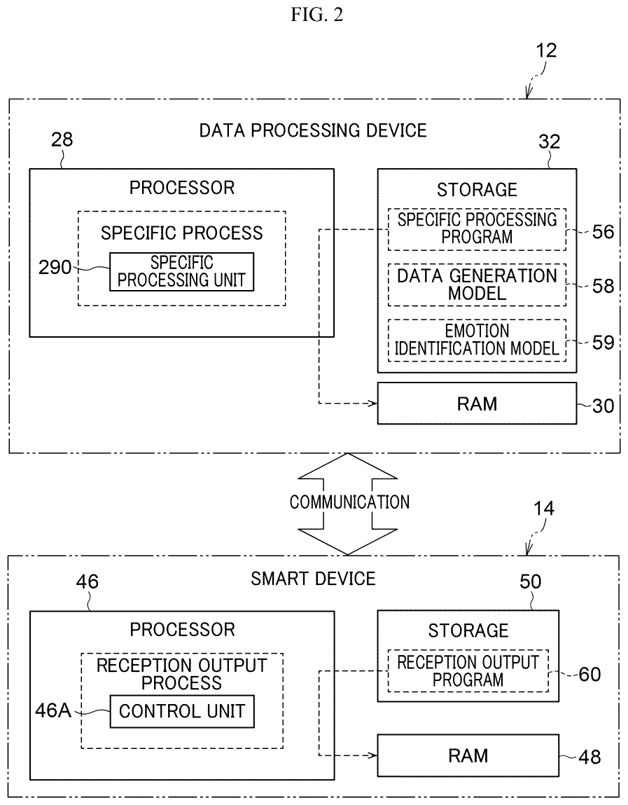

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computerized meal recommendation and delivery systems typically separate preference collection, recipe generation, kitchen instruction, delivery routing, and feedback analysis into loosely coupled or manually configured components. In many cases, servers merely store static user preferences and invoke pre-defined recipe templates or simple rule-based logic, without dynamically adapting the prompt generation to a generative AI model or utilizing structured feedback to improve system-level performance. As a result, such systems often suffer from several technical problems.

First, existing systems generally fail to generate prompt sentences to a generative AI model in a manner that systematically incorporates both explicit user preferences and accumulated feedback-derived preference parameters. This leads to unstable or suboptimal interaction between the application logic and the generative AI model, where the server cannot reliably obtain structured recipe information aligned with machine-understood constraints. Consequently, the server requires additional ad hoc parsing, error handling, or manual intervention, which increases processing overhead and degrades throughput and latency. Second, in many architectures, feedback from users regarding taste or delivery quality is stored as unstructured text and simple ratings, with little or no automated learning loop that feeds back into subsequent prompt generation. This prevents the system from updating internal preference parameters in a technically meaningful way and from improving the quality of generated recipes or delivery plans over time. The lack of a closed feedback loop at the processor level yields inefficient utilization of computational resources and limits personalization accuracy.

Third, conventional systems often treat delivery routing as an external function, not tightly integrated with the recipe generation pipeline and user preference learning. As a result, the server does not jointly optimize kitchen scheduling, route computation, and user satisfaction metrics. This fragmented processing induces redundant data transfers between heterogeneous components, repeated computations of route information, and inconsistent state management, thereby reducing overall system efficiency and scalability.

Fourth, there is a technical challenge in consistently obtaining machine-readable, structured recipe information from generative models that are inherently free-form text generators. Without carefully designed prompt sentences that specify output formats and embed learned preference parameters, the server cannot reliably parse or validate the generative output. This causes failures in downstream processes such as automatic generation of cooking instruction information and route optimization based on recipe attributes.

Accordingly, there is a need for an improved computer-implemented system in which a processor orchestrates: (i) generation of structured prompt sentences for a generative AI model based on both current user inputs and machine-learned preference parameters; (ii) automatic generation of structured recipe information and cooking instruction information from the generative AI output; (iii) integrated route optimization using delivery destination information; and (iv) machine learning-based analysis of user feedback to update internal preference parameters. Such a system should reduce manual configuration, minimize parsing errors, improve utilization of computing resources, and provide a technically improved end-to-end pipeline that enhances personalization accuracy and operational efficiency.

The specific processing by the specific processing unit 290 of the data processing device 12 in Example 1 is realized by the following means.

The present invention provides a server comprising a processor configured to receive, via an information processing terminal, preference information and feedback information regarding food from a user; to acquire, from a storage unit, the preference information and past feedback information of the user; to generate, based on the acquired information, a prompt sentence in a natural language including an output format specification to be input to a generative AI model; to input the prompt sentence to the generative AI model and obtain, from the generative AI model, structured recipe information including cooking procedure information and ingredient information corresponding to a preference of the user; to generate, based on the structured recipe information, cooking instruction information indicating contents of cooking work to be performed by a cooking apparatus, and to transmit the cooking instruction information to an information processing terminal installed in a cooking facility; to execute route optimization processing to determine, based on location information of a delivery destination, a delivery order and a movement route, and to transmit delivery instruction information including the determined delivery order and movement route to a delivery information processing terminal; and to analyze, by using a machine learning algorithm, feedback information including an evaluation of the recipe information and a delivery, thereby updating a parameter representing the preference of the user and reflecting the updated parameter in generation of a subsequent prompt sentence. This enables a technically improved end-to-end computer-implemented pipeline in which the server reliably obtains structured outputs from the generative AI model, reduces parsing and coordination overhead across recipe generation, kitchen control, and logistics routing, and continuously adapts internal preference parameters based on machine learning, thereby improving system efficiency, scalability, and personalization accuracy in comparison with conventional systems.

The term “processor” refers to one or more hardware-based processing units, such as a central processing unit or a graphics processing unit, and associated control circuitry configured to execute instructions for performing the functions described herein.

The term “server” refers to an electronic computing apparatus including at least one processor, a storage unit, and a communication interface, configured to provide processing, storage, and communication services to one or more external terminals over a communication network.

The term “information processing terminal” refers to an electronic device, such as a smartphone, tablet, personal computer, or dedicated terminal, including a processor, a display unit, an input unit, and a communication interface, configured to exchange data with the server and present information to a user.

The term “preference information” refers to data representing a user's tastes or requirements regarding food, including at least one of a desired type of cuisine, a desired flavor intensity, a dietary restriction, an allergy condition, or a portion size.

The term “feedback information” refers to data generated after provision of a meal or a delivery service, including at least one of a rating value, an evaluation of taste, an evaluation of quantity, an evaluation of delivery quality, or a free-text comment provided by a user.

The term “storage unit” refers to a hardware storage device, such as a non-volatile memory, a magnetic disk, or a solid-state drive, or a combination thereof, configured to store data including preference information, feedback information, recipe information, and program instructions.

The term “prompt sentence” refers to a sequence of characters expressed in a natural language, optionally including an instruction regarding an output format, provided as an input to a generative AI model to cause the generative AI model to generate a response.

The term “generative AI model” refers to a trained artificial intelligence model, such as a neural network-based language model, configured to generate text data including at least recipe information in response to a prompt sentence.

The term “recipe information” refers to structured or semi-structured data representing a preparation of a meal, including at least ingredient information and cooking procedure information.

The term “ingredient information” refers to data representing components used to prepare a meal, including at least a name of each ingredient, and optionally a quantity and a unit associated with each ingredient.

The term “cooking procedure information” refers to data representing a sequence of cooking operations to be performed to prepare a meal, including at least an order of steps, and optionally time information, temperature information, or device setting information.

The term “cooking instruction information” refers to data generated by the server based on recipe information, indicating contents of cooking work to be performed by a cooking apparatus or by staff in a cooking facility, including at least ingredient handling instructions and step-by-step operation instructions.

The term “cooking apparatus” refers to a device used in a cooking facility to perform at least one cooking operation, such as heating, boiling, frying, baking, or mixing, including at least one of a stove, an oven, a cooker, or a similar machine.

The term “cooking facility” refers to a location equipped with at least one cooking apparatus and at least one information processing terminal, where food is prepared according to cooking instruction information.

The term “location information” refers to data representing a destination to which a meal is to be delivered, including at least one of an address, a geographic coordinate, or other positional data.

The term “position information” refers to geographic coordinate data representing a position on a map, including at least one of a latitude, a longitude, or other coordinate system values corresponding to a delivery destination.

The term “route optimization processing” refers to computational processing in which a delivery order and a movement route are determined based on at least time information and distance information for one or more delivery destinations so as to optimize a performance measure such as total travel time or total travel distance.

The term “delivery instruction information” refers to data indicating at least one of a delivery order, a movement route, or timing information, provided from the server to a delivery information processing terminal to control or assist delivery of meals.

The term “delivery information processing terminal” refers to an information processing terminal operated by delivery personnel or mounted on a delivery vehicle, configured to receive delivery instruction information from the server and present delivery guidance.

The term “machine learning algorithm” refers to a computational procedure, implemented by software executed by the processor, that adjusts model parameters based on training data including feedback information, to infer or update a parameter representing a user's preference.

The term “parameter representing the preference of the user” refers to a numerical or categorical value maintained by the server, derived from at least feedback information, that quantifies an aspect of the user's taste or behavior, such as a preferred spiciness level, a preferred saltiness level, or a preference for a particular cuisine type.

The term “structured recipe information” refers to recipe information formatted according to a predefined data structure, such as a set of key-value pairs or a hierarchical data format, in which individual elements including ingredients and cooking steps are clearly identifiable by machine processing.

The term “output format specification” refers to an instruction contained in a prompt sentence that designates a required structure or representation of a response generated by the generative AI model, such as a list structure, a table structure, or a data format with specific fields.

In one embodiment, a server, one or more terminals, and a plurality of information processing devices cooperate to implement the present invention.

### A. Hardware and Software Configuration

A server includes at least one processor, a memory, a non-volatile storage unit, and a network interface. The processor is, for example, a multi-core central processing unit or a combination of a central processing unit and a graphics processing unit. The memory is, for example, a volatile memory such as a dynamic random access memory. The storage unit is, for example, a solid-state drive or a magnetic disk device. The network interface is configured to connect the server to a communication network such as the Internet.

The server executes an operating system such as a general-purpose server operating system, and application software implemented in a programming language such as C++, Java, or Python. The server further executes a database management program to control a relational database, and a middleware program or library to communicate with a generative AI model. In one embodiment, the server executes an HTTP server component, an API gateway component, a prompt generation component, a generative AI interface component, a recipe management component, a route optimization component, and a machine learning component.

A terminal includes at least one processor, a memory, a display, an input device such as a touch panel, and a wireless or wired communication interface. The terminal is, for example, a smartphone, a tablet computer, or a personal computer. The terminal executes an operating system suitable for mobile or desktop devices, and an application program providing graphical user interfaces for preference input, order confirmation, and feedback input. A cooking facility includes at least one cooking apparatus and at least one information processing terminal. The cooking apparatus includes, for example, an electric cooker, an oven, a stove, or a heating device equipped with a microcontroller and a control interface for receiving setting values such as temperature and time. The information processing terminal in the cooking facility is configured as a terminal similar to the above, and is used to display cooking instruction information to staff and, in some embodiments, to control settings of the cooking apparatus.

A delivery information processing terminal includes at least one processor, a GPS module, a display, and a communication interface. The delivery information processing terminal executes a program to present delivery instruction information and navigation guidance. In some embodiments, the generative AI model is executed on a physically separate computing system, such as an AI service platform connected to the server via the network. In other embodiments, the generative AI model is executed on the same server or on a dedicated inference machine including one or more graphics processing units.

### B. Data Structures and Modules

The server stores, in the storage unit, at least a user table, a preference table, a feedback table, a recipe table, a delivery table, and a model parameter table.

The user table stores user identifiers and basic user attributes. The preference table stores explicit preference records including a user identifier, cuisine type, desired spiciness level, desired saltiness level, allergy information, and other preference attributes. The feedback table stores, per order, a user identifier, ratings for taste, quantity, and delivery, and free-text comments. The recipe table stores structured recipe information including a recipe identifier, a title string, a list of ingredients represented as records of ingredient name, quantity value, and unit string, and a list of cooking procedure steps represented as ordered text strings. The delivery table stores delivery destination data including address strings and corresponding geographic coordinates. The model parameter table stores, for each user, preference parameters learned through machine learning, such as numerical preferred spiciness level, numerical preferred saltiness level, and preference vectors for cuisine categories.

The server logically divides functions into modules. A preference acquisition module manages reception and storage of user preference information. A prompt generation module constructs prompt sentences in a natural language suitable for input to the generative AI model. A generative AI interface module handles communication with the generative AI model. A recipe validation and storage module verifies and stores generated recipe information. A cooking instruction generation module converts recipe information into cooking instruction information including device-specific parameters. A route optimization module computes delivery order and movement routes. A feedback analysis module executes a machine learning algorithm to update user preference parameters.

The generative AI model is, in one embodiment, a neural network language model having a transformer architecture. The model includes an embedding layer that maps input tokens of a prompt sentence into continuous vectors, a plurality of self-attention layers, and a final projection layer that outputs probability distributions over tokens. Model parameters include weight matrices for attention and feed-forward sublayers, and are stored as floating point values. The generative AI model is trained, in advance, on a corpus including recipe texts and cooking instructions. The server does not re-train the entire generative AI model at runtime, but the server controls the input prompt sentence in order to adjust outputs.

The feedback analysis module employs a separate machine learning model implemented on the server. In one embodiment, the machine learning model is a gradient boosting regression model that predicts a user satisfaction score from recipe features and past feedback. In another embodiment, the machine learning model is a shallow neural network with fully connected layers. The machine learning model uses as features at least numerical values representing ingredient quantities, normalized spiciness scale, normalized saltiness scale, and vector representations of cuisine types. The model parameters are updated by minimizing a loss function such as mean squared error between predicted satisfaction and actual feedback ratings, using an optimization method such as stochastic gradient descent or gradient boosting.

### C. Operation of the Generative AI Model and Prompt Sentences

The server uses the prompt generation module to construct a prompt sentence based on explicit preference information and learned preference parameters. The prompt generation module generates a text string by concatenating template segments and variable segments. The template segments define a natural language description and an output format specification. The variable segments include values retrieved from the preference table and the model parameter table.

For example, when the user prefers spicy dishes and has a peanut allergy, the server generates a prompt sentence such as:

“User preferences: cuisine=Indian, spiciness=high, saltiness=medium, allergies=peanuts. Generate one dinner recipe that exactly matches these preferences. Provide ingredients with precise quantities and step-by-step instructions. Output the result as a structured list of ingredients followed by a numbered list of cooking steps.”

In another example, when the feedback analysis module has learned that the user prefers more saltiness than explicitly indicated, the server generates a prompt sentence such as: “User explicit preferences: cuisine=Indian, spiciness=high, allergies=peanuts. Learned profile: user consistently prefers slightly more saltiness than average. Generate a dinner recipe that reflects these preferences. Ensure the dish is peanut-free and has above-average saltiness. Provide a title, a list of ingredients with quantities and units, and detailed step-by-step cooking instructions.”

The server transmits the prompt sentence to the generative AI model through the generative AI interface module. The generative AI interface module encapsulates the prompt sentence into a request structure including model identification and generation parameters such as temperature and maximum output length.

The generative AI model receives the prompt sentence and processes it using the transformer architecture. The embedding layer transforms each token into a vector. Subsequent self-attention layers iteratively compute attention-weighted combinations of token embeddings using learned weight matrices. The model then outputs tokens corresponding to ingredient lists and cooking steps, respecting the output format specification because the prompt sentence includes explicit instructions for structure.

The server receives the generated text from the generative AI model and passes it to the recipe validation and storage module. This module parses the text based on the known structure indicated in the prompt sentence. Because the server caused the generative AI model to output ingredients as a list and steps as a numbered sequence, the parsing process becomes deterministic and efficient. The module converts the parsed data into internal recipe records and stores them in the recipe table.

This combination of prompt sentence design and deterministic parsing improves computing efficiency and reliability compared with conventional systems. The server reduces the need for complex and error-prone natural language parsing by constraining the generative outputs at the source. As a result, the server saves CPU cycles, reduces memory usage for error handling structures, and improves throughput under load.

### D. Machine Learning-Based Feedback Analysis and Preference Parameter Update

The server uses the feedback analysis module to transform sparse, heterogeneous feedback information into compact preference parameters. The server retrieves, from the feedback table and the recipe table, data required to construct a training dataset. Each training example includes recipe feature values and corresponding feedback ratings. Recipe feature values include, for example, normalized quantities of specific spices, total salt content, cooking time, and categorical attributes such as cuisine type. The server pre-processes textual comments by, for example, extracting occurrences of phrases like “too salty” or “not spicy enough” using pattern matching, and converts them into numerical indicators.

The feedback analysis module applies a machine learning algorithm, such as gradient boosting. The module computes, for each training example, a prediction of user satisfaction and an error value between the prediction and the actual rating. The module then updates model parameters by fitting decision trees or adjusting neural network weights to reduce the loss function.

The server aggregates model outputs per user to compute preference parameters. For example, the server computes, for each user, a preferred spiciness offset as the average difference between optimal recipe spiciness predicted by the model and baseline recipe spiciness. The server stores these preference parameters in the model parameter table. This processing is not a simple storage or averaging of feedback, but a structured learning procedure that captures relationships between recipe features and user satisfaction. Because the server uses model-based extraction of parameters rather than manual rule tuning, the system can handle a high-dimensional feature space and adapt to complex preference patterns. Furthermore, by maintaining compact numerical parameters instead of raw feedback histories, the server reduces storage requirements and accelerates subsequent prompt generation, contributing to improved computing performance.

### E. Integration with Cooking Apparatus and Delivery Routing

The server generates cooking instruction information from the structured recipe information. In one embodiment, the server converts cooking procedure information into device-specific command sets. For example, when a step indicates “bake at 180 degrees for 20 minutes,” the server generates control parameters setting temperature and time for an oven. When the cooking apparatus exposes a communication interface, such as a programmable logic controller interface or an application programming interface, the terminal in the cooking facility transmits these settings to the cooking apparatus. The cooking apparatus then autonomously sets internal timers and temperature controls based on digital signals.

By mapping recipe steps to control parameters for cooking apparatus, the server reduces the need for human translation of textual instructions into device settings, decreases the risk of human error, and increases consistency across executions. Moreover, because the recipe information is structured, the mapping operation is a deterministic transformation, which can be implemented as table-driven logic inside the cooking instruction generation module. This leads to predictable timing and resource utilization in the server.

The route optimization module of the server receives address information of delivery destinations and converts these addresses into geographic coordinates using geocoding algorithms. The module constructs a graph representation of a road network, with nodes representing positions and edges representing possible segments of travel, each associated with distance and estimated travel time. The module applies, for example, a shortest path algorithm or a vehicle routing problem algorithm to compute optimal delivery sequences and routes.

The server then embeds the computed delivery order and movement routes into delivery instruction information, which includes waypoints and expected arrival times. The delivery information processing terminal receives this information and, in some embodiments, uses a navigation software component to guide delivery personnel. The integration of route computation with order and recipe data allows the server to schedule cooking and delivery consistently, reducing idle times and preventing delays.

### F. Technical Effects and Computer-Technology Improvements

The server improves computer technology in several specific ways.

First, the server improves interaction with a generative AI model by generating prompt sentences that encode both user preferences and learned parameters, along with explicit output format specifications. This improves the determinism and parseability of generative outputs. As a result, the server performs fewer ambiguous parsing operations, resulting in reduced CPU time and memory consumption. The server thereby increases throughput and reduces latency in recipe generation workflows.

Second, the server uses a machine learning algorithm to transform large volumes of raw feedback into compact preference parameters. This leads to improved caching and data locality, because subsequent operations need only access small parameter vectors instead of full feedback histories. The reduction in data footprint improves access times, and the use of model-based parameter extraction increases the accuracy of personalization relative to simple rule-based or average-based methods.

Third, the server integrates recipe information with control parameters for cooking apparatus and with route optimization computations. The server thereby forms a pipeline from user input to device control and logistics routing that is executed by a consistent set of software modules and data structures. This integration reduces redundant computations and network round trips that would otherwise be necessary if separate services handled recipes, scheduling, and routing independently. The server's modules share structured data, which prevents repeated conversions between incompatible formats and reduces overall communication load.

Fourth, the server uses AI-related processing in a manner that is not a mere automation of human decision making. The generative AI model is constrained and guided by prompt sentences that encode machine-learned preference parameters and output format rules. Humans would typically not express preferences in such a structured way for machine parsing. The machine learning algorithm uses numerical features and error functions to optimize internal parameters, which is distinct from human intuition and heuristics. These computational methods enable pattern detection and adaptation at a scale and precision that manual configuration cannot achieve.

Fifth, the use of specific network and storage structures, such as separate tables for user preferences, feedback, recipes, deliveries, and model parameters, and the use of algorithmic modules such as route optimization and machine-learning-based preference inference, results in improved data management. The server avoids redundant storage of similar information and is able to update and query data with fewer and more efficient operations. This leads to reduced storage overhead and improved query performance.

### G. Variations and Alternative Embodiments

In a first variation, the generative AI model is replaced by another neural network language model or a sequence-to-sequence model trained on different corpora, as long as the server can construct prompt sentences and parse structured outputs. The server may adjust the template and format of prompt sentences to suit different models, but maintains the principle of embedding learned preference parameters into natural language instructions and format specifications.

In a second variation, the machine learning algorithm used in the feedback analysis module is replaced by a matrix factorization model or a collaborative filtering model. The server still computes preference parameters per user, such as latent factor values, and uses them to influence prompt sentences. The underlying loss function may be changed from mean squared error to another differentiable or piecewise differentiable function, but the effect of reducing prediction error and improving preference estimation remains.

In a third variation, the route optimization module uses different graph algorithms or heuristic methods, such as simulated annealing or genetic algorithms, to compute approximate solutions for delivery routes. The structure of the delivery instruction information and its integration with recipe data and preference parameters is maintained.

In a fourth variation, the cooking apparatus control is performed indirectly by displaying numeric settings on the terminal in the cooking facility, which then are manually input into devices by staff. Even in this case, the server still converts recipe information into specific numerical setting suggestions, thereby reducing cognitive load and error risks for staff and enabling consistent cooking results.

In a fifth variation, the server executes part of the machine learning operations on a dedicated accelerator, such as a hardware accelerator for matrix multiplication. The offloading of computation to specialized hardware further reduces processing time and energy consumption.

In all embodiments and variations, the server, the terminal, and the user act as distinct entities. The server performs primary data processing, model interaction, optimization, and control-related computations. The terminal performs user interaction functions, data input, confirmation, and, in some cases, control of on-site devices. The user provides preference input and feedback, and consumes meals prepared and delivered as a result of the server's processing. Through these interactions and technical configurations, the present invention provides an implementation that others skilled in the art can realize using conventional computing hardware and software components, while achieving improved technical performance and reliability in comparison with conventional systems.

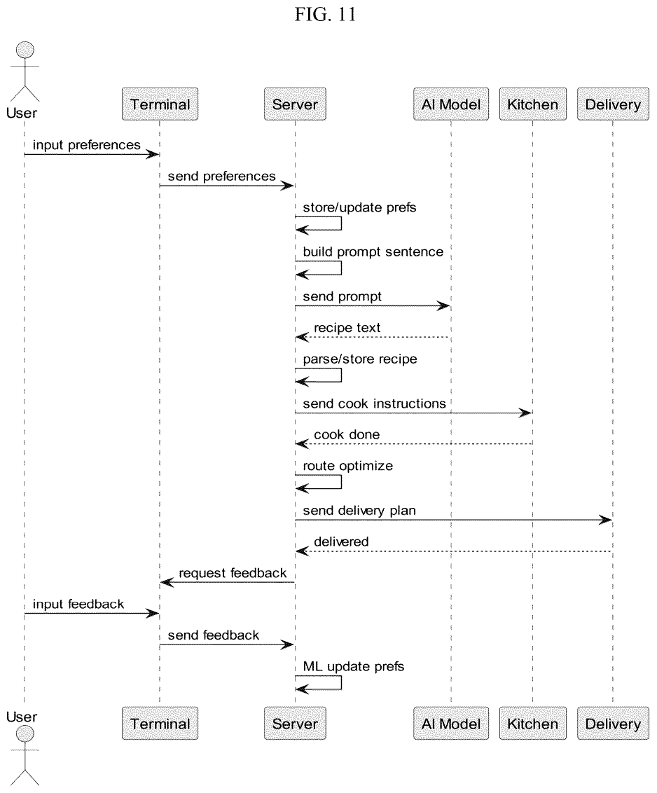

The following describes the processing flow using FIG. 11.

### Step 1:

User operates the Terminal to input food preference information.

User selects, on a graphical user interface of the Terminal, at least cuisine type, desired spiciness level, desired saltiness level, allergy information, and portion size.

Terminal receives touch or click events as input and updates internal variables representing each preference item.

Terminal validates the input by checking that required fields are not empty and that selected allergy items are within a predefined list.

Terminal generates, as output, a structured preference record in memory, such as a key-value map containing user identifier, cuisine type, spiciness level, saltiness level, and allergy list.

### Step 2:

Terminal transmits preference information to the Server.

Terminal takes the structured preference record from Step 1 as input.

Terminal serializes the record into a text-based format, such as JSON, and encapsulates it into an HTTP request body.

Terminal sends the HTTP request over a network to a predefined API endpoint exposed by the Server.

Server receives, as output of this step, a network message that includes the serialized preference information.

### Step 3:

Server parses and stores the user preference information.

Server takes as input the HTTP request containing the serialized preference information from Step 2.

Server uses an HTTP server component to parse the request, extract the JSON body, and convert it into an internal data structure, such as a dictionary or object.

Server validates that the user identifier exists and that preference values are within predetermined ranges.

Server executes database operations (insert or update) to store the preference data into a preference table, thereby updating the user's preference profile.

Server outputs an updated preference profile in persistent storage and, optionally, a success response message to the Terminal.

### Step 4:

Server acquires explicit preference information and learned preference parameters.

Server takes as input a user identifier associated with an upcoming recipe generation request. Server queries the preference table to retrieve explicit preference information and queries a model parameter table to retrieve learned preference parameters representing the user's inferred tastes.

Server combines explicit attributes (such as cuisine type and allergy list) with learned parameters (such as preferred spiciness offset and preferred saltiness offset) into a composite internal structure.

Server outputs a unified preference representation that will be used in subsequent prompt generation.

### Step 5:

Server generates a prompt sentence for the generative AI model.

Server takes as input the unified preference representation from Step 4.

Server selects a template for a prompt sentence and fills placeholders with the explicit preference values and learned parameters.

Server appends an output format specification that instructs the generative AI model to output structured recipe information.

Server creates, as output, a prompt sentence expressed in natural language, such as: “User explicit preferences: cuisine=Indian, spiciness=high, allergies=peanuts. Learned profile: user consistently prefers slightly more saltiness than average. Generate a dinner recipe that reflects these preferences. Ensure the dish is peanut-free and has above-average saltiness. Provide a title, a list of ingredients with quantities and units, and detailed step-by-step cooking instructions.”

### Step 6:

Server transmits the prompt sentence to the generative AI model and receives generated text.

Server takes as input the prompt sentence from Step 5.

Server constructs a request payload including the prompt sentence, model identifier, and generation parameters such as temperature and maximum token count.

Server sends this payload to an endpoint of the generative AI model over the network.

Generative AI model processes the prompt sentence internally and returns a generated text response that is expected to comply with the requested format.

Server receives, as output, a text string containing ingredients and cooking procedure information.

### Step 7:

Server parses and validates the generated recipe information.

Server takes as input the generated text from the generative AI model received in Step 6.

Server applies parsing rules based on the known output format, such as splitting the text into a title section, an ingredient list section, and a cooking steps section.

Server extracts ingredient names, quantities, and units, and extracts each cooking step as an ordered string.

Server verifies constraints, such as exclusion of ingredients listed in the user's allergy information and presence of required fields.

Server outputs structured recipe information in an internal representation, including a title string, a collection of ingredient records, and a collection of ordered step records.

### Step 8:

Server stores the structured recipe information.

Server takes as input the structured recipe representation from Step 7 and a corresponding user identifier and order identifier.

Server constructs database insert commands to store recipe attributes into a recipe table and to associate the recipe with the user and the order.

Server writes the data into persistent storage and verifies completion of the write operation.

Server outputs a stored recipe entry with a unique recipe identifier that can be referenced by other modules.

### Step 9:

Server generates cooking instruction information for the cooking facility.

Server takes as input the structured recipe information and the capabilities of cooking apparatus available at a target cooking facility.

Server converts human-readable cooking steps into device-oriented instructions, such as numeric temperature settings, time durations, and operation modes, based on mapping rules that relate textual actions (“bake”, “fry”, “boil”) to apparatus parameters.

Server aggregates these device-specific settings with the recipe title and ingredient list into a cooking instruction data structure.

Server outputs cooking instruction information that indicates both human-readable guidance for staff and machine-readable parameters for cooking apparatus.

### Step 10:

Server transmits cooking instruction information to a terminal in the cooking facility.

Server takes as input the cooking instruction data structure from Step 9 and the network address of the cooking facility terminal.

Server serializes the instruction data and sends it via an HTTP request or another communication protocol to the facility's terminal.

Terminal in the cooking facility receives and deserializes the data, preparing it for display and potential apparatus control.

Server outputs a successful transmission status and an associated order state indicating that cooking instructions have been issued.

### Step 11:

Terminal in the cooking facility displays and optionally forwards cooking instructions.

Terminal takes as input the cooking instruction information received from the Server in Step 10.

Terminal renders, on a display, the recipe title, ingredient list with quantities, and step-by-step instructions for human staff.

Terminal optionally translates device-related parameters into control signals to be sent to connected cooking apparatus via a local interface, such as setting oven temperature or timer values.

Terminal outputs visual guidance for staff and, when applicable, control actions applied to cooking apparatus.

### Step 12:

Server performs route optimization based on delivery destination information.

Server takes as input one or more delivery destinations associated with orders, each including at least an address or geographic coordinate.

Server converts addresses into position information, such as latitude and longitude, using a geocoding process if needed.

Server constructs a graph or matrix representing travel distances and times between possible stops and applies a route optimization algorithm to compute an efficient delivery order and movement route.

Server outputs a delivery plan that includes an ordered list of destinations, associated paths, and estimated arrival times.

### Step 13:

Server generates and transmits delivery instruction information.

Server takes as input the delivery plan from Step 12 and metadata for each order, such as order identifiers and user identifiers.

Server encapsulates waypoints, route segments, and expected times into delivery instruction information formatted for a delivery information processing terminal.

Server sends this information via a network to the delivery terminal used by delivery staff.

Server outputs a record indicating that delivery instructions have been dispatched and updates order states to a status indicating that delivery is in progress.

### Step 14:

Terminal for delivery staff presents navigation and delivery details.

Terminal takes as input the delivery instruction information from the Server in Step 13.

Terminal displays, on a map or list interface, the delivery order, each destination address, and estimated arrival times, and may invoke a navigation module to provide turn-by-turn directions.

Terminal allows delivery staff to mark each delivery as completed or failed and to record basic comments if necessary.

Terminal outputs status updates for each delivery, which are sent back to the Server as structured messages.

### Step 15:

Server updates delivery status and triggers feedback requests.

Server takes as input delivery status messages received from the delivery terminal in Step 14.

Server parses the messages to identify delivered orders and updates corresponding entries in a delivery table and an order table to a status indicating successful completion.

Server checks configuration rules and, when an order is marked as delivered, generates a trigger signal requesting user feedback.

Server outputs updated delivery and order records and feedback request notifications addressed to the corresponding user terminals.

### Step 16:

User inputs feedback information via the Terminal.

User receives a notification or prompt on the Terminal requesting an evaluation of the meal and delivery.

User enters ratings for aspects such as taste, spiciness, saltiness, portion size, and delivery timeliness, and optionally types a free-text comment.

Terminal validates rating ranges and checks text against simple content rules, then packages the ratings and comment into a structured feedback record.

Terminal outputs the structured feedback data and transmits it to the Server as a network message.

### Step 17:

Server stores and preprocesses feedback information.

Server takes as input the structured feedback data from Step 16.

Server parses the incoming data, associates it with the correct user and order identifiers, and writes the feedback into a feedback table.

Server optionally performs preliminary preprocessing, such as normalizing rating scales and extracting simple indicators from text comments (for example, flags for “too spicy” or “too salty”).

Server outputs stored feedback entries and intermediate feature values that are ready for further machine learning processing.

### Step 18:

Server performs machine learning-based analysis and updates user preference parameters.

Server takes as input aggregated feedback records and associated recipe features drawn from the recipe table for multiple past orders.

Server constructs a training dataset where each instance contains feature vectors representing recipe attributes (such as normalized spice quantities, salt quantities, cuisine type encodings) and target values representing user satisfaction ratings.

Server applies a machine learning algorithm, such as gradient boosting or a neural network, to compute updated model parameters that minimize a loss function measuring the error between predicted and actual satisfaction.

Server derives, from the trained model, updated preference parameters for each user, including numerical values representing adjusted preferred spiciness and saltiness and other taste-related metrics, and stores them in the model parameter table.

Server outputs refreshed preference parameters that capture learned user tastes and are used as inputs to subsequent prompt sentence generation, thereby closing the feedback loop and improving future recipe personalization.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computer-implemented food recommendation and delivery systems typically rely on static rule-based filtering or simple collaborative filtering techniques that operate on coarse user attributes and past orders. In such systems, server-side processing often treats user preference data, delivery status data, and user feedback data as independent information streams, without tightly integrating them into a single, dynamically updated user model. As a result, existing systems suffer from several technical problems.

First, conventional servers generally construct menu recommendations using fixed query conditions against a database of items, without generating machine-interpretable prompt sentences that can fully express complex user preference structures to a generative AI model. In these systems, user interfaces merely collect preference fields, and the back-end processing converts them into simple database filters. This limits the expressiveness of user preference representation and prevents the server from leveraging modern generative models to dynamically generate high-quality recipe information or menu description information tailored to nuanced user tastes.

Second, existing feedback processing pipelines typically store user ratings and free-text comments as raw data, or at most as simple aggregate scores, without performing systematic natural language processing and machine learning to extract structured satisfaction indices for multiple taste attributes. In such architectures, servers frequently fail to transform noisy feedback streams into stable, machine-learned user preference patterns that can continuously refine future recommendations. This leads to technical inefficiencies: recommendation quality degrades over time, and each new feedback item does not effectively update the underlying model parameters or profiles.

Third, many current systems handle delivery tracking and menu generation as separate modules. Delivery status is updated and displayed to the user, but the information is not looped back into the preference modeling pipeline. Therefore, the server does not utilize temporal delivery history or user reactions to delivery experiences as signals for adaptive learning. This results in a fragmented architecture in which the server cannot globally optimize recommendation generation, cooking instruction generation, and delivery instruction generation.

Fourth, known systems often treat the generative AI model as a black-box text generator, without providing a standardized, machine-generated prompt sentence that has been optimized by learned user profiles and structured satisfaction indices. This ad-hoc use of generative models causes technical issues, such as unstable output quality, inconsistent personalization, and the inability to systematically improve prompt generation logic over time based on learned patterns.

Thus, there is a need for a computer-implemented system, and particularly for server-side processing logic, that (i) integrates user preference information, delivery history information, and feedback information into a coherent data model; (ii) automatically generates and updates prompt sentences for a generative AI model based on learned preference patterns; (iii) transforms unstructured textual feedback into structured satisfaction indices through natural language processing; and (iv) uses a machine learning model to continuously refine both prompt generation processing and candidate menu selection processing. Such a system would improve the functioning of the underlying computer technology by enabling more efficient data utilization, more adaptive model updating, and more accurate and personalized generation of recipe information and menu description information in real time.

The specific processing by the specific processing unit 290 of the data processing device 12 in Application Example 1 is realized by the following means.

The present invention provides a server comprising a processor configured to provide a user interface through an information processing terminal to receive, from a user, preference information and feedback information related to meals and to acquire the preference information and the feedback information from the information processing terminal, to analyze the acquired preference information and the acquired feedback information as user information to extract meal constraint conditions, flavor tendencies, and ingredient preferences of the user and to generate, in accordance with an extraction result, a prompt sentence to be input to a generative AI model, to input the generated prompt sentence and candidate menu information associated with the user information to the generative AI model and to cause the generative AI model to generate recipe information or menu description information suitable for preferences of the user, to generate and transmit cooking instruction information including cooking target items and cooking conditions to a cooking facility based on the recipe information or the menu description information and to generate and transmit delivery instruction information including a delivery route and an estimated arrival time to a delivery facility, to update a delivery status based on position information and delivery state information acquired from the delivery facility and to notify the updated delivery status to the information processing terminal, to input the feedback information and history information related to the delivery status to a machine learning model to cause the machine learning model to learn preference patterns and evaluation tendencies for each user and to update processing for generating the prompt sentence and processing for selecting the candidate menu information based on a learning result, and to update and store user profile information, used for generation of the recipe information or the menu description information in subsequent operations, based on the learning result and the user information. This enables the server to technically improve recommendation accuracy and system adaptability by transforming heterogeneous user interaction data into structured, machine-learned profiles that automatically optimize prompt sentence generation for the generative AI model and candidate menu selection, thereby enhancing computer-based processing efficiency, stabilizing generative output quality, and providing dynamically personalized cooking and delivery instructions in a unified architecture.

The term “system” refers to an integrated combination of hardware and software components, including at least one processor and associated storage and communication resources, configured to execute the claimed functions.

The term “processor” refers to one or more hardware processing units, such as a central processing unit or other computation circuitry, capable of executing instructions to perform the described operations.

The term “information processing terminal” refers to an electronic device, such as a mobile device, a tablet device, or a general-purpose computer, that presents a user interface and communicates with the system over a communication network.

The term “user interface” refers to a software-controlled interaction mechanism, including graphical elements, input fields, and display components, that enables a user to input data and receive information via the information processing terminal.

The term “preference information” refers to data indicating a user's tastes, constraints, and choices related to meals, including, for example, food categories, taste strengths, ingredient preferences, and dietary restrictions.

The term “feedback information” refers to data indicating a user's evaluation of provided meals or services, including, for example, rating values, free-text comments, and other post-consumption responses.

The term “user information” refers to a data set including at least the preference information and the feedback information associated with a particular user, and may further include historical usage data and profile data.

The term “meal constraint conditions” refers to limitations or requirements related to meals, such as dietary restrictions, allergen avoidance, nutritional limits, or prohibited ingredients.

The term “flavor tendencies” refers to patterns or preferences in a user's desired taste characteristics, including, for example, preferred levels of spiciness, saltiness, sweetness, and sourness.

The term “ingredient preferences” refers to user-specific indications of favored or disfavored ingredients, including preferred ingredients to be included and ingredients to be excluded from meals.

The term “prompt sentence” refers to a structured natural-language instruction or query generated by the processor for input to a generative AI model, the instruction or query expressing user-related conditions and requirements to guide content generation.

The term “generative AI model” refers to a machine learning model configured to generate output data, such as text, in response to input data, including but not limited to large language models that generate recipe information or menu description information from prompt sentences and associated parameters.

The term “candidate menu information” refers to data representing multiple selectable meal items, each item having associated attributes such as ingredients, taste properties, and preparation conditions, from which recommended items may be selected.

The term “recipe information” refers to structured data describing at least one meal, including information about required ingredients, quantities, and preparation or cooking steps.

The term “menu description information” refers to natural-language text or structured description that explains or describes one or more meals, including characteristics such as flavor profile, ingredients, and serving style.

The term “cooking instruction information” refers to data transmitted to a cooking facility that specifies which items are to be prepared and how they are to be prepared, including at least cooking target items and cooking conditions.

The term “cooking target items” refers to specific meal items selected for preparation for a user, each item being associated with recipe information.

The term “cooking conditions” refers to parameters that define how cooking is to be performed, including, for example, temperature, time, seasoning levels, and inclusion or exclusion of certain ingredients.

The term “cooking facility” refers to a physical location or equipment environment, such as a kitchen or food preparation station, configured to receive cooking instruction information and prepare meals accordingly.

The term “delivery instruction information” refers to data transmitted to a delivery facility that specifies delivery-related parameters, including a delivery route and an estimated arrival time.

The term “delivery route” refers to a path or sequence of locations used to transport prepared meals from the cooking facility to a user's delivery destination.

The term “estimated arrival time” refers to a predicted time at which a delivery of a prepared meal is expected to reach the user's delivery destination.

The term “delivery facility” refers to an entity or arrangement, including delivery personnel and associated devices, configured to receive delivery instruction information and perform transportation of prepared meals.

The term “delivery status” refers to state information indicating a current condition of a delivery process, including stages such as pending, in preparation, in transit, and delivered.

The term “position information” refers to data indicating a geographic location, including, for example, coordinates representing the position of a delivery resource or a delivery object.

The term “delivery state information” refers to non-positional data that represents the current progress or condition of a delivery, such as start time, completion status, or delay indicators.

The term “machine learning model” refers to a computational model constructed using machine learning techniques, configured to learn patterns from data and produce outputs such as predictions or classifications based on learned parameters.

The term “preference patterns” refers to learned representations of recurring relationships between user characteristics and choices, indicating how a user tends to select or evaluate meals.

The term “evaluation tendencies” refers to learned patterns in how a user provides feedback or ratings, including biases towards certain attributes or consistent evaluation behaviors.

The term “learning result” refers to model parameters, internal representations, or derived metrics produced by training or updating the machine learning model on collected user information and feedback.

The term “user profile information” refers to a stored data structure associated with a user, containing aggregated and learned information such as preference patterns, constraint conditions, and derived satisfaction indices, and used for future content generation and recommendation.

The term “natural language processing” refers to a set of computational techniques for analyzing and processing human language data, including tokenization, syntactic analysis, semantic analysis, and extraction of structured features from text.

The term “semantic information” refers to meaning-related data extracted from text, representing concepts, attributes, and relations, such as taste orientation, nutritional constraints, and cooking conditions contained in a prompt sentence or feedback.

The term “input format for the generative AI model” refers to a representation, such as a token sequence or structured data encoding, that is compatible with the generative AI model's interface and can be processed by the model to generate output.

The term “evaluation value information” refers to quantitative assessment data provided by a user, such as numeric ratings or scores indicating satisfaction with meals or services.

The term “free-text information” refers to unstructured text data entered by a user, such as open-ended comments describing impressions, complaints, or requests.

The term “order history information” refers to records of past orders associated with a user, including ordered items, times, and any associated evaluations or outcomes.

The term “feature extraction processing” refers to computational operations that transform raw data, particularly free-text information, into structured features or indices suitable for input to a machine learning model.

The term “satisfaction indices” refers to numerical or categorical indicators that quantify a user's level of satisfaction with respect to specific attributes, such as spiciness level, salt level, or fat content.

In one embodiment, a server is implemented as a network-connected computing apparatus including at least one processor, a main memory, a non-volatile storage device, and a network interface. The server executes an operating system and one or more application programs that implement the functions described in the claims. The server is connected via a communication network to one or more terminals operated by users and to one or more devices or systems located at cooking facilities and delivery facilities.

A terminal is implemented as a mobile communication device, such as a smartphone or a tablet, or as a general-purpose computer. The terminal includes a display, an input device such as a touch panel, a wireless communication module, a processor, and a memory. The terminal executes an application program that presents a user interface for acquisition of preference information and feedback information from a user, and that communicates such information to the server using, for example, HTTP or HTTPS over a packet-switched network.

A user interacts with the terminal to provide preference information, feedback information, and order-related information. The user selects options or inputs natural-language text through the user interface. The terminal converts the user's selections and text into structured data, such as key-value pairs, and transmits the structured data to the server.

The server uses specific hardware and software components to perform data processing and data computation. For example, in one embodiment, the server uses a relational database management system, such as a general-purpose relational database, running on the same hardware or on a separate storage server, to persistently store user information, menu information, recipe information, delivery information, and model parameters. The server uses an application framework, such as a general web-application framework, to route incoming requests, parse data, and call internal modules.

The server maintains specific data structures to efficiently implement the claimed functions. For example, the server stores user information in a user profile table, where each record includes a user identifier, encoded meal constraint conditions, encoded flavor tendencies, encoded ingredient preferences, and one or more learned preference vectors. The server stores feedback information in a feedback table, where each record includes a user identifier, an order identifier, an evaluation value, a free-text comment, and one or more extracted feature values. The server stores menu information in a menu table, where each record includes an item identifier, ingredient attributes, taste attributes, nutritional attributes, and cooking condition attributes. The server may additionally store delivery history information in a delivery table, where each record includes route attributes, time attributes, and delivery state attributes.

The server analyzes acquired preference information and feedback information using a combination of deterministic processing and machine learning processing. The server first applies deterministic parsing and normalization to the raw data. For example, the server maps user-selected options, such as “spicy,” “mild,” “low fat,” and “no dairy,” to numerical codes or binary flags in a structured feature vector. The server further performs natural language processing on free-text information, such as comments and narrative preference descriptions, using text-processing libraries. The server tokenizes the text, removes stop words, applies lemmatization or stemming, and computes word embeddings or other vector representations. In one embodiment, the server uses a neural network-based natural language encoder to convert free-text information into dense vectors. The server may use, for example, a bidirectional recurrent neural network or a transformer-based encoder, comprising an embedding layer, multiple hidden layers with attention mechanisms, and an output projection layer. The server trains this encoder on a corpus of food-related text and user feedback so that the encoder maps similar preference expressions to similar vectors.

The server extracts meal constraint conditions, flavor tendencies, and ingredient preferences by applying trained classification or regression models to the structured feature vectors and text vectors. For example, the server may use a multi-label classifier that outputs probabilities for constraints (allergies, forbidden ingredients), a regressor that outputs preferred intensity levels for attributes such as spiciness and saltiness, and a classifier that outputs likelihood of preference or aversion for particular ingredients. These models may be implemented using gradient-boosted decision trees, fully connected neural networks, or a combination thereof. Based on such extraction results, the server generates a prompt sentence that encodes user-specific conditions in natural language, in a structure that is optimized for input to a generative AI model. The server uses a prompt-generation module that constructs the prompt sentence according to a predefined template, inserting specific values derived from the user profile and the extracted attributes. For example, the server may generate a prompt sentence such as:

“Generate three dinner recipes that are very spicy, contain no dairy products, and avoid cilantro, suitable for a user who prefers Asian-style cuisine and moderate fat content. Include a short description for each recipe.”

The server may generate alternative prompt sentences depending on the type of content to be produced. For instance, the server may supply a prompt sentence such as:

“Based on the following user feedback on delivered meals, summarize the user's evolving preferences regarding spiciness, saltiness, and oiliness, and propose updated preference settings that will improve future menu recommendations.”

In another embodiment, the server generates a prompt sentence such as:

“Design a feedback processing workflow that analyzes user ratings and comments on delivered meals, updates a machine learning preference model, and outputs features that can be used for next-time personalized menu recommendations in a food delivery system.”

The server uses these prompt sentences not only to generate recipes or menu descriptions, but also to refine internal parameters or to simulate workflow variants.

The server inputs the generated prompt sentence and candidate menu information to a generative AI model. In one embodiment, the generative AI model is implemented as a large neural network having a transformer architecture, including multiple self-attention layers, feed-forward layers, and normalization layers. The server transmits the prompt sentence and structured context (such as a list of candidate dishes with attributes) to the generative AI model over an internal interface or an external network interface. The generative AI model outputs text sequences representing recipe information or menu description information. The server parses the output sequences to identify recipe components, such as ingredient lists and stepwise instructions, or to extract description segments to be presented to the user.

The server further constructs cooking instruction information based on the generated recipe information or menu description information. The server determines cooking target items, including dish identifiers and any modifications required to satisfy the user's constraints, such as substitution of ingredients to avoid allergens. The server determines cooking conditions, such as target cooking times, temperature ranges, and seasoning intensities, by mapping abstract instructions in the recipe information to machine-readable parameters. The server transmits these parameters to a cooking facility through a network interface. As a result, the system does not merely convey human-readable text, but controls machine-readable data structures that can be interpreted by automated equipment or kitchen display systems at the cooking facility.

The server constructs delivery instruction information including a delivery route and an estimated arrival time. The server may call a route calculation service, which computes a path between the cooking facility and the user's delivery destination based on geographic data and traffic data. The server receives a polyline or a series of coordinate points representing the delivery route and a time estimate for the route. The server stores these in the delivery table and transmits an abstracted form of the route and the estimated arrival time to a delivery facility, such as a delivery management system or a driver's terminal. Because the server uses structured route representations, the server can efficiently update and compare routes, and can adjust the estimated arrival time as new position information is received.

The server updates delivery status based on position information and delivery state information acquired from the delivery facility. The server receives periodic updates containing current coordinates and state flags, such as “picked up,” “on route,” or “delivered.” The server computes distance-to-destination values by applying a distance calculation algorithm to the current position and destination coordinates. The server stores updated status codes and remaining distances in the delivery table. The server notifies updated delivery status to the terminal by sending concise status messages or push notifications. By encoding status in a compact, indexed format and transmitting only changes, the server reduces communication load and improves responsiveness.

The server uses a machine learning model to learn preference patterns and evaluation tendencies from feedback information and delivery history information. In one embodiment, the machine learning model is implemented as a multi-task neural network that receives, as input, a concatenated feature vector including user profile features, menu item features, delivery context features (such as time-of-day and delay metrics), and feedback features (such as rating values and extracted sentiment scores). The neural network includes an input layer, multiple hidden layers with nonlinear activation functions, and separate output heads for predicting future satisfaction scores and for updating specific preference dimensions. The server trains this neural network using supervised learning, with a loss function that combines squared error for rating predictions and cross-entropy loss for classification of attribute preferences.

During training, the server computes gradients of the loss function with respect to network weights, applies an optimization algorithm such as stochastic gradient descent or an adaptive gradient method, and updates the weights stored in the model parameter store. The server may perform mini-batch training on a scheduled basis, or online training where each new feedback item is used to incrementally update the model. The server may also perform data augmentation, for example by perturbing attribute values within ranges or by generating synthetic text variants using the generative AI model, to improve robustness of the preference model. By performing these operations in the server, the system improves prediction accuracy and reduces the number of user interactions needed to converge to stable preference profiles.

The server updates user profile information based on learning results. For each user, the server stores a current preference vector that encodes attribute weights for spiciness, saltiness, oiliness, sweetness, ingredient categories, and nutritional preferences. The server also stores a confidence value for each attribute, indicating how reliably the attribute is inferred from past data. After training, the server receives updated weights and confidence values from the machine learning model and writes them back to the user profile table. This structure allows the server to quickly compute candidate menu scores by performing vector operations between the user preference vector and menu item feature vectors, thereby improving computational efficiency compared to repeated rule-based filtering.

The above-described architecture and processing flow provide technical improvements beyond simple automation of human tasks. The server uses specialized data structures, machine learning algorithms, and prompt generation logic to reduce computation time and memory usage when generating recommendations at scale. For example, by precomputing and storing user preference vectors and embedding menu items into the same feature space, the server can apply efficient matrix multiplication or nearest-neighbor search to select candidate menu items, which reduces latency when many users simultaneously request recommendations.

Further, by converting unstructured feedback text into structured satisfaction indices and storing them as numerical attributes, the server enables faster and more accurate updates of preference vectors. The server does not merely replace manual reading of comments, but performs quantitative modeling that improves the precision of subsequent predictions and reduces variance in user satisfaction scores. As a result, the server can generate more accurate and stable prompt sentences for the generative AI model, which in turn produces more consistent recipe information and menu description information.

The generative AI model integration also leads to technical benefits. The server provides prompt sentences that are not arbitrary, but are generated by a deterministic pipeline that uses machine-learned user profiles and structured constraints. This reduces the need to repeatedly adjust prompts at runtime and decreases the number of interactions between the server and the generative AI model, thereby reducing communication overhead and computational load on the model inference engine. The server can cache frequently used prompt patterns and associated outputs, and can detect when user preferences have not changed significantly, in which case existing cached recommendations may be reused.

The system also provides technical advantages in data management and consistency. Because the server stores user information, recipe information, menu information, and delivery information in normalized tables with specific keys, the server can enforce referential integrity and can efficiently execute joins when generating views of user history. This allows the server to quickly reconstruct longitudinal behavior patterns and to adjust recommendations in near real-time. The use of structured tables and defined primary and foreign keys improves maintainability and reduces error rates compared to ad-hoc storage of logs.

In a variant embodiment, the server implements a modular architecture in which distinct modules handle user interface communication, preference extraction, prompt generation, generative model interaction, cooking instruction generation, delivery management, and learning. Each module exposes a defined interface and operates on clearly defined data structures. The server may deploy these modules on separate hardware nodes or virtual machines to scale each function independently. This modular design facilitates optimization of specific components, such as placing the generative AI model interaction module on hardware with specialized accelerators, while keeping the main application logic on general-purpose servers.

In another embodiment, the server applies rule-based processing prior to machine learning inference. For example, the server enforces hard safety constraints (such as allergen avoidance) through deterministic rules in a constraint engine, while using the machine learning model only for soft preference dimensions (such as taste intensity). This hybrid approach ensures that certain classes of errors are eliminated entirely, thus improving safety and reliability. At the same time, the machine learning component optimizes subjective attributes, yielding higher satisfaction without sacrificing constraint compliance.

The server can also be configured to handle different types of generative AI models. In one variant, the server uses a text-only model that outputs recipe descriptions. In another variant, the server uses a multimodal model that can generate both text and structured tags, allowing the server to directly receive attribute annotations for generated recipes. The server may adjust the prompt sentence accordingly, for example, by requesting explicit tags for allergen content or nutritional values, thereby improving the granularity of subsequent recommendations.

The described system is tightly integrated with real-world device control and logistics. The server does not merely generate abstract suggestions but generates machine-readable cooking instruction information that may be consumed by kitchen display systems or automated cooking devices, and machine-readable delivery instruction information that may be consumed by navigation systems or delivery scheduling software. This coupling ensures that improvements in the preference modeling and prompt generation logic have direct technical impact on routing efficiency, preparation timing, and resource allocation, leading to reduced delays, decreased waste of ingredients, and more balanced utilization of cooking and delivery capacity.

Through these concrete configurations, data structures, and algorithms, the server, the terminal, and the user cooperate to implement a system in which generative AI models and machine learning models are used to improve core computer functions, including data processing efficiency, storage efficiency, and communication efficiency, and to provide a tightly integrated, technically optimized pipeline from user interaction to cooking and delivery control.

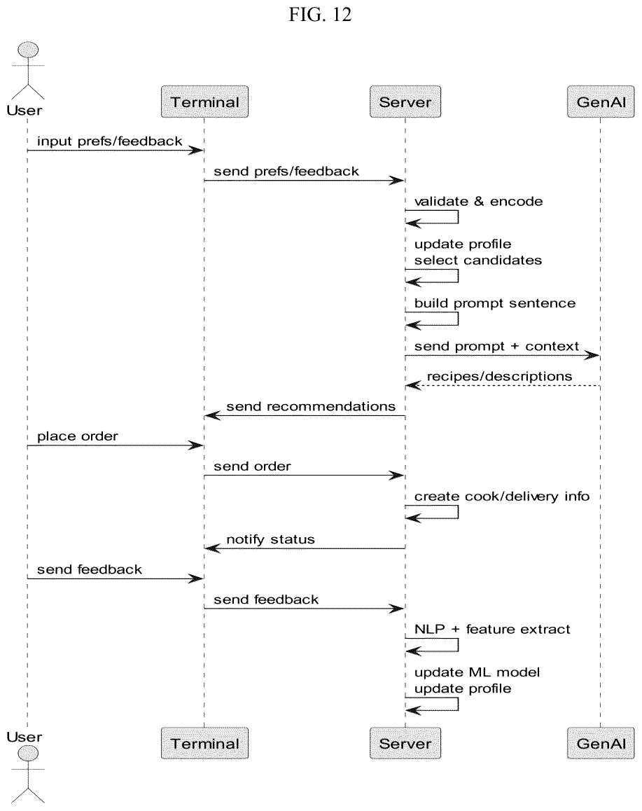

The following describes the processing flow using FIG. 12.

### Step 1:

User operates the terminal to input preference information and feedback information.

User selects items on a graphical user interface, such as preferred cuisines, spiciness level, disliked ingredients, and dietary constraints, and may also type free-text comments describing desired tastes.

Terminal receives these user interactions as raw UI events and converts them into structured key-value data.

Input: User's touch operations, selections, and typed text.

Output: A structured data object containing fields such as user identifier, categorical preferences, numerical taste levels, and free-text preference descriptions.

### Step 2:

Terminal packages the structured data into a request message and transmits it to the server.

Terminal serializes the structured data into a machine-readable format, attaches metadata such as timestamps and application version, and sends the message over a network connection.

Input: Structured preference and feedback data generated in Step 1.

Output: A network request containing the structured data, delivered via a communication protocol to the server.

### Step 3:

Server receives the request and validates the incoming data.

Server parses the message, checks that required fields such as user identifier and at least one preference attribute are present, verifies that values fall within allowed ranges, and discards malformed records.

Input: Network request containing structured user data from the terminal.

Output: A validated internal representation of user preference information and feedback information, suitable for further processing.

### Step 4:

Server normalizes and encodes the validated data into internal feature vectors.

Server maps categorical values (for example, cuisine types and taste labels) to numerical codes, scales numerical values such as intensity levels into normalized ranges, and encodes binary constraints such as allergy flags.

Server applies natural language processing to any free-text content, performs tokenization, removes stop words, and generates a dense vector representation using a text encoder.

Input: Validated structured fields and raw text comments.

Output: One or more numerical feature vectors representing meal constraint conditions, flavor tendencies, ingredient preferences, and textual nuances.

### Step 5:

Server extracts explicit meal constraint conditions, flavor tendencies, and ingredient preferences from the feature vectors.

Server applies trained classification and regression models to the feature vectors, computing probabilities for each constraint (for example, allergen avoidance), estimating preferred intensity values for attributes such as spiciness and saltiness, and inferring preference scores for individual ingredients.

Input: Encoded feature vectors from Step 4.

Output: A set of extracted attribute values, including constraint flags, numerical taste parameters, and ingredient preference scores, assembled into an intermediate user attribute structure.

### Step 6:

Server updates a persistent user profile with the extracted attributes.

Server reads the existing record for the user from a profile storage, merges newly extracted attributes with previously stored attributes, and resolves conflicts using rules or weighted averages that prioritize recent, high-confidence data.

Server writes updated values, including current preference vectors and constraint flags, back to the profile storage.

Input: Intermediate user attribute structure from Step 5 and existing profile record.

Output: An updated user profile record in persistent storage, containing current constraint conditions and a refined preference vector.

### Step 7:

Server selects candidate menu items based on the updated user profile.

Server queries a menu database for items, filters out items that violate hard constraints such as allergens and prohibited ingredients, and computes similarity scores between the user preference vector and feature vectors for each remaining item using distance or dot-product operations.

Server sorts the items according to similarity or predicted satisfaction score and selects a subset as candidate menu information.

Input: Updated user profile record and menu item feature records.

Output: A ranked list of candidate menu items with associated attributes to be used for generative processing.

### Step 8:

Server generates a prompt sentence for a generative AI model using the user profile and candidate menu attributes.

Server constructs a natural-language instruction according to a predefined template, inserting specific user constraints and preferences as variables, and optionally appending a summary of candidate dishes.

For example, the server may generate a prompt sentence such as:

“Generate three dinner recipes that are very spicy, contain no dairy products, and avoid cilantro, suitable for a user who prefers Asian-style cuisine and moderate fat content. Include a short description for each recipe.”

Input: User profile attributes and candidate menu attributes.

Output: A prompt sentence that encodes user-specific conditions and context in natural language.

### Step 9:

Server sends the prompt sentence and structured context to a generative AI model and obtains generated content.

Server formats the prompt sentence and candidate menu information into an input representation compatible with the generative AI model, transmits the input over an internal or external interface, and receives generated text as output.

Input: Prompt sentence from Step 8 and candidate menu information from Step 7.

Output: Generated recipe information and menu description information in textual form, associated with specific dishes or newly proposed meal options.

### Step 10:

Server parses the generated content and converts it into structured recipe information and menu description information.

Server splits the generated text into segments corresponding to individual recipes, extracts ingredient lists, cooking steps, and descriptive phrases using text parsing and pattern recognition, and maps these elements into structured fields.

Input: Raw generated text output from the generative AI model.

Output: Structured recipe records and descriptive text fields suitable for display and for generation of machine-readable instructions.

### Step 11:

Server creates cooking instruction information for a cooking facility.

Server selects one or more structured recipes or modified menu items, identifies cooking target items, and converts qualitative descriptions such as “very spicy” into parameter values such as seasoning intensity levels.

Server assembles a message that includes dish identifiers, ingredient quantities, cooking times, temperature ranges, and any user-specific modifications.

Input: Structured recipe information from Step 10 and user constraint parameters from the profile.

Output: Cooking instruction information containing cooking target items and cooking conditions, formatted for consumption by a kitchen system.

### Step 12:

Server transmits the cooking instruction information to a cooking facility and records the associated state.

Server sends the instruction message to a kitchen display system or control system over a communication channel, and updates an order record in storage to indicate that preparation has been requested.

Input: Cooking instruction information from Step 11.

Output: A transmitted instruction message and an updated order state stored in the server.

### Step 13:

Server generates delivery instruction information including a delivery route and an estimated arrival time.

Server obtains origin and destination coordinates, calls a routing component or external routing service to compute a path and travel time, and encodes the route as a sequence of waypoints or a compact representation.

Server combines the route representation with time estimates and identifiers to form a delivery instruction message.

Input: Order details, user delivery address, and current location of the cooking facility.

Output: Delivery instruction information specifying a route and an estimated arrival time.

### Step 14:

Server transmits the delivery instruction information to a delivery facility and initializes delivery tracking.

Server sends the route and timing data to a delivery management system or a driver's terminal, and creates a delivery record in storage, setting the initial delivery status and timestamps.

Input: Delivery instruction information from Step 13.

Output: A transmitted delivery message and an initialized delivery record with status fields set to an initial value.

### Step 15:

Delivery facility or driver terminal periodically sends position information and delivery state information back to the server.

Terminal on the delivery side collects coordinates from positioning hardware and updates state flags such as “en route” or “near destination,” and transmits these in structured messages to the server.

Input: Device sensor readings and operational state changes at the delivery facility.

Output: Network messages containing position information and delivery state information, addressed to the server.

### Step 16:

Server updates delivery status and computes refined estimated arrival times based on incoming delivery data.

Server receives position updates, calculates distances to the destination using geographic computations, and applies prediction logic to adjust the estimated arrival time.

Server writes updated status codes, distances, and refined estimates into the delivery record.

Input: Position information and delivery state information from Step 15, and existing delivery record.

Output: An updated delivery record with current status, remaining distance, and revised estimated arrival time.

### Step 17:

Server notifies the terminal of the updated delivery status.

Server generates concise status messages, including textual notifications and numerical time-to-arrival values, and sends them to the terminal using a messaging channel or push notification service.

Terminal receives these notifications, updates a tracking display, and optionally renders a map view indicating the latest position.

Input: Updated delivery record from Step 16.

Output: Status messages delivered to the terminal and updated visual information on the terminal's user interface.

### Step 18:

User receives the prepared meal and submits feedback through the terminal.

User opens a feedback interface, selects a rating value, and may provide free-text comments such as “Spiciness was perfect, but it was a bit too oily.”

Terminal records this information, associates it with the relevant order, and prepares it for transmission.

Input: User's rating selections and typed comments.

Output: Structured feedback data containing identifiers, numerical ratings, and textual comments.

### Step 19:

Terminal transmits the structured feedback data to the server.

Terminal constructs a request containing the feedback fields, attaches identifiers for user and order, and sends the message over the network.

Input: Structured feedback data from Step 18.

Output: A feedback message received by the server.

### Step 20:

Server stores the feedback information and preprocesses the text to extract features.

Server writes the feedback data into a feedback storage with fields for ratings, comments, and timestamps.

Server then applies natural language processing to the comment text, tokenizes the text, applies sentiment analysis, and derives satisfaction indices for attributes such as spiciness, saltiness, and oiliness.

Input: Feedback message from Step 19.

Output: Stored feedback records and computed numerical feature values representing attribute-specific satisfaction.

### Step 21:

Server updates the machine learning model using the new feedback features.

Server forms training samples by combining user profile features, menu item features, delivery context features, and satisfaction indices derived from the comment.

Server computes loss values based on prediction errors between model outputs and observed ratings, calculates gradients, and updates model weights.

Input: Training samples constructed from stored data and newly computed features.

Output: Updated model parameters that encode refined preference patterns and evaluation tendencies.

### Step 22:

Server regenerates or refines the user profile based on the updated model.

Server passes the user's aggregated feature data through the updated machine learning model to obtain a new preference vector and updated confidence values for each attribute.

Server modifies the user's profile record accordingly, replacing or adjusting prior attribute weights with new values that better reflect learned preferences.

Input: Updated model parameters from Step 21 and historical user data.

Output: A refined user profile containing an updated preference vector and associated attribute confidences.

### Step 23:

Server adjusts subsequent prompt sentence generation and candidate menu selection using the refined user profile.

Server updates parameters in the prompt-generation module to emphasize attributes with higher confidence and to de-emphasize attributes with low or conflicting evidence, so that future prompt sentences more accurately represent the user's tastes.

Server also modifies scoring functions used in candidate menu selection to align with newly learned attribute weights.

Input: Refined user profile from Step 22.

Output: Updated configuration for prompt sentence templates and scoring logic that will influence future recommendation cycles.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented meal recommendation and ordering systems primarily focus on displaying menus and collecting simple orders, without deeply integrating user preference learning, recipe generation, cooking control, delivery scheduling, and secure pickup into a unified technical workflow. In such systems, server-side components often treat these processes as independent subsystems. As a result, data related to user preferences, generated recipes, cooking progress, delivery status, and pickup authentication are fragmented across different applications and data structures. This fragmentation leads to several technical problems, including increased server load for redundant data processing, latency caused by multiple uncoordinated API calls, inefficient route planning due to incomplete status information, and complex synchronization logic between disparate services.

Further, in many existing systems, the use of generative AI models is limited to ad hoc content generation, and is not systematically integrated with structured preference data and operational constraints such as kitchen capacity, preparation time, and delivery windows. Generative models are often invoked with manually crafted prompts that do not fully reflect a user's historical behavior or current operational conditions. This causes suboptimal utilization of computing resources and yields outputs that frequently require additional server-side post-processing, thereby increasing computation time and network traffic.

Moreover, typical server implementations do not tightly couple user feedback and natural language evaluation with a continuous learning loop. Textual feedback is often stored as unstructured logs and is not effectively used to refine models or adjust downstream control decisions regarding cooking instructions, delivery scheduling, and authentication token management. This leads to repeated execution of similar recommendation and planning calculations without leveraging prior feedback, thereby wasting processing cycles and limiting the personalization quality that could otherwise be achieved with better model updates.

Additionally, conventional delivery and pickup flows usually rely on simple, manually configured status flags and static authentication codes. These mechanisms do not exploit end-to-end digital state management from recipe generation to locker unlock control. As a result, server components must frequently poll multiple systems, reconcile inconsistent states, and handle failures using costly error-handling routines. This increases the complexity of the server logic, makes real-time tracking difficult, and degrades system responsiveness. Accordingly, there is a need for an improved computer-implemented system that (i) integrates user preference learning, generative AI model prompting, and recipe generation into a cohesive server-side process, (ii) automatically derives cooking instruction data and delivery plan data from the generated recipe information and real-time operational data, and (iii) manages authentication information and locker unlock control within the same server-controlled workflow. Such a system should reduce redundant data transformations, minimize cross-service latency, and improve the technical efficiency and reliability of the overall meal preparation, delivery, and pickup pipeline.

The specific processing by the specific processing unit 290 of the data processing device 12 in Example 2 is realized by the following means.

The present invention provides a server comprising a processor configured to provide, on an information processing device, an interface for receiving input information related to meals from a user; analyze, on the information processing device, the input information and past usage history information and evaluation information of the user to derive preference information of the user regarding meals; generate, based on the preference information, a prompt sentence for instructing a generative AI model to generate a recipe or meal menu corresponding to the preference information; input the prompt sentence to the generative AI model and acquire recipe information based on the preference information of the user; output, based on the recipe information, cooking instruction information to a cooking facility, the cooking instruction information including meal content to be prepared and a preparation completion time; generate delivery plan information based on cooking completion information from the cooking facility and pickup location information and pickup time information specified by the user, and output the delivery plan information to a mobile terminal so as to determine a delivery route and delivery time; generate authentication information used for receiving the meal stored in a storage apparatus installed at the pickup location, transmit the authentication information to a user terminal, store the authentication information in association with the storage apparatus, determine validity of an input result of the authentication information transmitted from the storage apparatus, transmit response information for controlling unlocking of the storage apparatus to the storage apparatus when the authentication information is valid, and update a state of corresponding order information. This enables a unified, processor-implemented workflow that tightly couples preference learning, generative AI model interaction, operational instruction generation, delivery scheduling, and secure pickup control, thereby reducing redundant data processing, lowering communication overhead between subsystems, improving response latency, and enhancing overall computational efficiency and reliability of the meal recommendation, preparation, delivery, and pickup system.

The term “system” refers to a combination of one or more computing devices, storage devices, communication interfaces, and associated software components that cooperate to execute the processes described in the claims.

The term “processor” refers to a hardware data processing unit, such as a central processing unit or a microcontroller, capable of executing instructions stored in a memory to perform logical, arithmetic, control, and input/output operations.

The term “information processing device” refers to an electronic apparatus, such as a server, client terminal, or embedded controller, that includes at least a processor, a memory, and a communication interface, and that executes software to process digital data.

The term “user” refers to a human individual or an entity that operates a terminal device or interacts with the system in order to request, receive, or manage meal-related services.

The term “input information related to meals” refers to digital data received from the user, including at least meal preferences, menu selections, dietary restrictions, pickup locations, pickup times, and other order-related parameters.

The term “past usage history information” refers to stored digital records indicating previous interactions between the user and the system, including past orders, viewed items, accepted recommendations, and transaction timestamps.

The term “evaluation information” refers to feedback data provided by the user, including at least ratings, comments, and other qualitative or quantitative assessments regarding meals or services.

The term “preference information” refers to structured data representing inferred or explicit tendencies of the user regarding meals, including preferred attributes such as cuisine type, taste, ingredients, portion size, nutritional characteristics, or price range.

The term “prompt sentence” refers to a text string or equivalent structured representation that encodes instructions or contextual information for input to a generative AI model, so as to cause the model to generate a recipe or meal menu.

The term “generative AI model” refers to a parameterized computational model, such as a neural network or other machine learning model, configured to generate text or other structured content in response to a prompt sentence.

The term “recipe information” refers to data generated by the generative AI model that specifies at least the ingredients, preparation steps, cooking methods, and optionally nutritional or presentation details for a meal.

The term “meal menu” refers to a set of one or more meals or dishes proposed or selected for the user, including associated descriptions, categories, and optional configuration parameters.

The term “cooking facility” refers to a physical environment or installation, such as a kitchen or food preparation area, equipped with cooking equipment and optionally with a terminal device for receiving digital cooking instructions.

The term “cooking instruction information” refers to digital data transmitted to the cooking facility that specifies what meal content is to be prepared, how it is to be prepared, and by when it should be completed.

The term “meal content to be prepared” refers to one or more dishes, items, or portions defined by type, quantity, and configuration, which are to be produced by the cooking facility.

The term “preparation completion time” refers to a time or time interval by which the cooking facility is expected to finish preparing the specified meal content.

The term “cooking completion information” refers to status data transmitted from the cooking facility to the server indicating that the preparation of the specified meal content has been completed or has reached a particular stage.

The term “pickup location information” refers to data indicating a physical site where the user is to receive a prepared meal, such as an address, facility identifier, or locker installation identifier.

The term “pickup time information” refers to data indicating a target time or time window during which the user intends or is scheduled to receive the prepared meal at the pickup location.

The term “delivery plan information” refers to data that defines at least a delivery route, a delivery sequence, and one or more scheduled delivery times for transporting meals from a cooking facility to one or more pickup locations.

The term “mobile terminal” refers to a portable electronic device, such as a smartphone or tablet, carried by delivery personnel or users, which includes a processor, a communication interface, and an input/output interface.

The term “delivery route” refers to an ordered path or sequence of waypoints, including at least one pickup location, along which a delivery vehicle or delivery person travels to deliver meals.

The term “delivery time” refers to a time or time window associated with arrival at a particular pickup location or with completion of a delivery task.

The term “storage apparatus” refers to a physical storage unit or locker that is configured to store a prepared meal and that includes a controllable lock mechanism and an interface for communication with the server.

The term “authentication information” refers to data used to verify that a user is authorized to retrieve a stored meal from the storage apparatus, including at least one of a code, token, identifier, or other verifiable credential.

The term “user terminal” refers to an electronic device operated by the user, such as a smartphone, tablet, or personal computer, that communicates with the server and displays information related to orders, authentication, and pickup.

The term “input result of the authentication information” refers to data representing authentication information that has been input at the storage apparatus, for example via a keypad, touch panel, or code scanner.

The term “response information for controlling unlocking of the storage apparatus” refers to data transmitted from the server to the storage apparatus to command or permit the lock mechanism to transition between locked and unlocked states.

The term “order information” refers to data representing a request by the user for one or more meals, including at least meal identifiers, quantities, associated recipe information, and associated status information.

The term “state of corresponding order information” refers to a status attribute or set of attributes associated with order information, indicating a processing stage such as ordered, in preparation, ready for delivery, at pickup location, or picked up.

The term “natural language processing technique” refers to a computational method or algorithm for analyzing, interpreting, or transforming human language text, including at least tokenization, parsing, classification, and feature extraction.

The term “feature quantities” refers to numerical or categorical values derived from raw data, representing properties or attributes used as inputs to a machine learning model.

The term “input content to the generative AI model” refers to data provided to the generative AI model for processing, including at least the prompt sentence and optionally additional context or parameters.

The term “learning process” refers to a computational procedure in which a model's parameters are updated based on training data, such as past usage history information and evaluation information, in order to improve prediction or generation performance.

The term “preference parameters” refers to adjustable values within a model or data structure that quantify aspects of a user's meal-related preferences, used to influence generation of prompts, recipes, and plans.

The term “updated preference parameters” refers to preference parameters that have been modified as a result of a learning process, reflecting newly incorporated usage history information or evaluation information.

In one embodiment, a server, one or more terminals, and a user cooperate to implement the claimed system for generating meal recipes using a generative AI model, controlling cooking and delivery operations, and managing secure pickup via a storage apparatus.

### A. Overall Hardware and Software Configuration

The server uses a computing platform that includes at least one processor, a main memory, a non-volatile storage device, and a network interface. The server runs an operating system such as a general-purpose server operating system and executes middleware and application software. The server uses a web application framework such as an HTTP server component and an application framework, a database management system such as a relational database engine, and an AI execution environment such as a machine learning framework capable of running a neural network-based generative AI model.

The terminal uses a portable computing device, such as a smartphone or tablet, equipped with a processor, a memory, a wireless communication interface, a display, and an input device such as a touch panel. The terminal executes a client application, such as a native mobile application, for interacting with the server. The terminal of delivery personnel additionally uses a location acquisition component, such as a GPS receiver, and a map rendering component for route guidance.

The user uses a user terminal to input meal-related information and to receive authentication information. The user operates a graphical user interface that is generated by the client application and that communicates with the server via a network using a secure protocol such as HTTPS.

A storage apparatus at a pickup location uses an embedded controller that includes a processor, memory, and a network interface. The storage apparatus is equipped with a lock actuator such as an electronic solenoid, a door sensor, and a user interface such as a keypad, a touch screen, or a code reader. The storage apparatus executes a control program that exchanges authentication-related data with the server and controls locking and unlocking based on responses from the server.

A cooking facility uses one or more terminals such as kitchen display terminals or industrial PCs that are connected to the server. The cooking facility executes a cooking management program that displays cooking instruction information received from the server and transmits cooking completion information back to the server.

### B. Server-Side Data Structures and Modules

The server uses specific data structures in the database to implement the claimed features. For example, the server uses a user table that stores a user identifier, profile attributes, and references to usage history and evaluation information. The server uses an orders table that stores an order identifier, a user identifier, recipe information identifiers, order status values, pickup location identifiers, and timestamps. The server uses a feedback table that stores an evaluation identifier, an order identifier, a rating value, and free-text evaluation information.

The server uses a user_preferences table that stores a user identifier and preference parameters such as weights for taste attributes, cuisine categories, spice level, and dietary constraints. The server uses a pickup_tokens table that stores an authentication token, an associated order identifier, a storage apparatus identifier, a validity period, and a usage status. The server uses a delivery_plans table that stores delivery plan information including a route sequence, estimated arrival times, and terminal identifiers.

The server decomposes its functionality into software modules. The server uses a user interface module to provide screens and API responses for receiving input information related to meals from the user. The server uses a preference analysis module to analyze input information, past usage history, and evaluation information, and to derive preference information. The server uses a prompt generation module to generate a prompt sentence that encodes preference information and operational constraints for input to a generative AI model. The server uses a generative model interface module to call an external or internal generative AI model, such as a transformer-based language model, and to obtain recipe information. The server uses a cooking instruction module to convert recipe information into structured cooking instruction information for the cooking facility. The server uses a delivery planning module to compute delivery plan information based on cooking completion information and pickup constraints. The server uses an authentication management module to generate authentication information, associate it with orders and storage apparatus identifiers, and verify input results received from the storage apparatus. The server uses an order state management module to update the state of corresponding order information in response to events received from terminals and the storage apparatus.

### C. Generative AI Model and Prompt Sentence Configuration

The server uses a generative AI model implemented as a neural network, such as a transformer architecture comprising an encoder portion and a decoder portion with multiple self-attention layers and feedforward layers. The generative AI model receives a tokenized sequence representing a prompt sentence and outputs a tokenized sequence representing recipe information. The server uses a tokenizer to convert text into token identifiers and uses an embedding layer of the generative AI model to convert token identifiers into continuous vectors. The server uses positional encodings and attention mechanisms to process the input sequence.

The server configures the generative AI model with a set of parameters that are optimized by training on a corpus of recipe texts and meal descriptions. During training, the server uses a loss function such as cross-entropy between predicted tokens and ground-truth tokens and uses an optimization algorithm such as stochastic gradient descent with adaptive learning rate. The server adjusts model weights using backpropagation based on gradients computed from the loss function. The server optionally augments training data by applying data augmentation techniques, such as synonym replacement or ingredient substitution patterns, to increase robustness.

For runtime inference, the server adjusts input content to the generative AI model based on user preference parameters. For example, the server encodes high weights for spicy cuisine and low weights for caloric content by appending specific descriptive phrases or control tokens to the prompt sentence. The server may generate a prompt sentence such as: “Generate a lunch recipe for a single adult that matches the following preferences: spicy taste, low sugar, no nuts, and suitable for delivery in 20 minutes. The recipe should list ingredients, quantities, and step-by-step cooking instructions.”

The server tokenizes this prompt sentence and supplies it to the generative AI model. The generative AI model outputs a sequence representing a recipe, which the server de-tokenizes into human-readable recipe information. By structurally encoding preference information and timing constraints into the prompt sentence, the server causes the generative AI model to produce recipes that are directly compatible with downstream cooking instruction generation and delivery planning, reducing the need for post-processing.

### D. Preference Analysis and Learning

The server uses the preference analysis module to transform raw usage history and evaluation information into numerical feature quantities. The server represents each meal as a feature vector including dimensions such as cuisine type, dominant flavor profile, ingredient categories, estimated preparation time, calorie level, and price. The server represents user interactions, such as orders and ratings, as samples linking user identifiers to meal feature vectors and feedback scores.

The server uses a preference learning algorithm such as matrix factorization, gradient boosting, or a neural network to estimate preference parameters for each user. In one embodiment, the server uses a neural network with an embedding layer for users and an embedding layer for meals, and a feedforward network that predicts a rating or preference score. The server defines a loss function such as mean squared error between predicted ratings and actual ratings and uses gradient-based optimization to update preference parameters and model weights.

The server periodically executes a learning process as a background task. The server retrieves recent usage history and evaluation information, constructs minibatches of samples, computes gradients of the loss function with respect to the preference parameters and model weights, and updates those parameters. The server then writes updated preference parameters to the user_preferences table. By executing this learning process periodically, the server ensures that later prompt sentences, cooking instruction information, and delivery plan information are generated based on preference parameters that reflect the latest user behavior.

### E. Cooking Instruction Information and Delivery Plan Generation

The server converts recipe information into a structured representation for the cooking facility. The server parses the recipe text into a data structure that separates ingredients, quantities, preparation steps, and timing information. The server maps text-based descriptions to standardized identifiers used in the cooking facility, such as ingredient codes and equipment usage codes. The server computes a preparation completion time based on the recipe's estimated cooking duration and the user's requested pickup time, and then embeds that completion time into cooking instruction information.

The server transmits cooking instruction information to a terminal in the cooking facility using a communication protocol such as HTTPS with a structured format such as JSON or another machine-readable format. The server includes in the cooking instruction information a recipe identifier, a list of dishes, required quantities, an earliest start time, and a latest completion time. The server records that cooking instruction information in the database with a link to the corresponding order.

When the cooking facility sends cooking completion information, the server uses the delivery planning module to generate delivery plan information. The server calculates feasible delivery windows by comparing the actual completion time with the user's pickup time constraints and with existing delivery tasks. The server computes candidate routes using a routing algorithm. In one embodiment, the server uses a shortest-path algorithm or a variant of a vehicle routing algorithm that receives as inputs the geographic coordinates of pickup locations, current location of the delivery terminal, and expected travel times from map-based data. The server generates a route sequence and predicted arrival times and sends delivery plan information to the delivery terminal.

By integrating real-time cooking completion information, preference-based timing constraints, and routing computation, the server reduces unnecessary recalculation of routes and minimizes network communication between independent services. The server maintains a coherent state machine for each order, which improves scheduling accuracy and reduces late deliveries.

### F. Authentication Information Management and Storage Apparatus Control

The server uses the authentication management module to generate authentication information for secure pickup at the storage apparatus. The server uses a cryptographically secure random number generator to create a one-time code or token. The server associates the token with the order and a specific compartment of the storage apparatus by storing it in the pickup_tokens table. The server defines a validity period that is aligned with the delivery plan and expected arrival of the user.

The server transmits the authentication information to the user terminal, for example by embedding the code in an API response to the client application and by sending a notification message. The user sees the authentication code or a representation of the code, such as a pattern that can be entered at the storage apparatus.

When the storage apparatus receives an input result of the authentication information from a user, the storage apparatus sends that input result to the server. The server performs validity checks by searching the pickup_tokens table for a matching token, verifying that the token is associated with the correct storage apparatus and order, and confirming that the token has not expired or been used. If the server determines that the authentication information is valid, the server generates response information that instructs the storage apparatus to unlock a specific compartment.

The storage apparatus receives the response information and actuates its lock mechanism by controlling a solenoid or another actuator. The storage apparatus monitors door open and close events via sensors and sends a pickup completion event to the server when the user retrieves the meal. The server updates the order state to indicate that pickup is complete and marks the authentication token as used.

This end-to-end digital control of the storage apparatus improves technical reliability over conventional static code systems because the server maintains synchronized state of tokens, storage compartments, and orders and can prevent code reuse and unauthorized access.

### G. Technical Effects and Improvement of Computer Technology

The server improves computer technology by tightly coupling generative AI-based recipe generation with structured operational data and device control in a way that reduces redundant computation and network communication. The server avoids generic “data retrieval-display” loops by operating on specific data structures and by using preference parameters and learned models directly to shape prompt sentences and to derive actionable instructions for downstream modules.

By encoding preference parameters and operational constraints into the prompt sentence, the server reduces the dimensionality of downstream processing. The generative AI model generates recipes that already satisfy timing and constraint conditions, thus reducing the amount of server-side filtering, rule-based adjustments, and recomputation that would otherwise be necessary. This decreases processing load on the server and reduces latency between user request and available recipe information.

By using a specific preference learning architecture and explicit feature quantities, the server improves prediction accuracy of user preferences. Higher accuracy enables the server to avoid repeated trial-and-error recipe generations and order cancellations. This reduces the number of interactions between the server and client terminals and lowers network traffic. The server's periodic learning process also allows updating preference parameters in batch, which is more efficient than recalculating preferences on-demand for each new request. By using structured data tables and a finite state representation of each order, the server simplifies synchronization between modules and reduces errors caused by inconsistent status updates. Clear state transitions such as “PENDING_COOKING,” “COOKED_READY_FOR_DELIVERY,” “AT_LOCKER_WAITING_FOR_PICKUP,” and “PICKED_UP” allow the server to implement non-ambiguous logic for routing, token generation, and storage apparatus unlocking. This reduces the need for error correction routines and manual interventions, thus improving robustness and reliability.

By integrating delivery planning with real-time cooking completion information, the server performs route optimization only when necessary and with accurate timing data, thereby improving computational efficiency. The server reduces redundant queries to external routing services by caching route segments and by using incremental adjustments to existing delivery plans. This reduces communication load and improves response times for delivery terminals. By implementing authentication management in coordination with order states and storage apparatus identifiers, the server reduces the risk of invalid unlock operations and enhances security without human supervision. The server uses validity periods and one-time tokens to ensure that codes are not reused across orders or time windows. This digital control mechanism is more technically sophisticated than manual key distribution and contributes to improved access control for unattended pickup locations.

### H. Alternative Embodiments and Variations

In another embodiment, the server uses a different type of generative AI model, such as a sequence-to-sequence model with recurrent units, rather than a transformer. The server still generates a prompt sentence encoding preference information and operational constraints and still derives recipe information from the model. The server can also adjust decoding strategies, such as using beam search with length penalties to balance detail and conciseness of recipe information.

In another embodiment, the server uses a hybrid rule-based and model-based approach. The server first filters candidate recipe templates based on user allergies and hard constraints and then uses the generative AI model to fill in detailed ingredient quantities and instructions. This reduces the search space and improves the stability of the generative process.

In another embodiment, the terminal of the user operates as a thin client and renders the user interface as a web application instead of a native application. The terminal still sends structured input information to the server and receives recipe information, cooking status, and authentication information. The server's internal data structures and state management are unchanged.

In another embodiment, the storage apparatus communicates indirectly with the server via a local gateway device. The gateway aggregates lock control commands and authentication input results for multiple storage compartments and relays them to the server. The server still determines validity of authentication information and generates response information for unlocking, using the same data structures for tokens and order states.

In yet another embodiment, the server extends the technical effects by logging performance metrics for each route, such as travel times and deviation from planned arrival times, and feeding these metrics into a learning process for route estimation. The server uses machine learning techniques to refine travel time predictions for specific routes and times of day, thereby improving the accuracy of delivery planning and further reducing delays and network calls to external routing services.

Through these embodiments, the server, terminal, and user operate within a system that is not limited to abstract data manipulation, but instead implements concrete control over cooking facilities, delivery flows, and storage apparatuses. The server's specific data structures, learning mechanisms, and generative AI model interfacing collectively improve computational efficiency, accuracy, and reliability compared with conventional systems that separate recommendation, ordering, delivery, and pickup into loosely coupled modules.

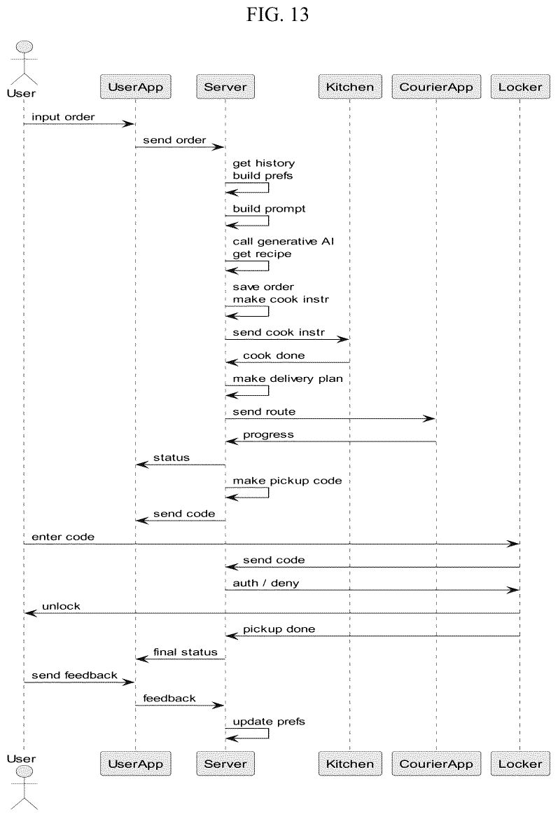

The following describes the processing flow using FIG. 13.

### Step 1:

User operates a user terminal to input meal-related information.

User selects menu items, specifies dietary preferences, enters pickup location information, and sets pickup time information through a graphical user interface.

Input: User provides textual and structured data such as preferred cuisine, allergies, desired time window, and location identifier.

Output: User terminal generates a structured request payload containing the meal-related input information and transmits it to the server via a network using a secure protocol.

### Step 2:

Server receives and validates the input information.

Server accepts the request payload from the user terminal at an application programming interface endpoint and parses the payload into internal data structures.

Input: Server receives the structured request including user identifier, selected menus or free-form preference text, pickup location identifier, and pickup time information.

Output: Server produces validated input records by checking required fields, verifying time formats, confirming existence of the location in a database, and rejecting or correcting inconsistent values. Server writes the validated data to temporary in-memory representations for further processing.

### Step 3:

Server retrieves past usage history information and evaluation information.

Server queries a database for records associated with the user identifier to obtain historical orders, ratings, and feedback text.

Input: Server takes the user identifier as a key and executes database retrieval operations on tables storing usage history and evaluation information.

Output: Server generates an aggregated user interaction dataset that includes a list of past orders, meal feature vectors, rating values, and textual review entries, which it stores in working memory.

### Step 4:

Server derives preference information from historical data.

Server processes the aggregated user interaction dataset using a preference analysis module that computes feature quantities and updates preference parameters.

Input: Server uses historical meal feature vectors, associated ratings, and textual evaluations.

Output: Server calculates numerical preference parameters such as weightings for cuisine types, spice level, ingredient inclusion or exclusion, and timing preferences, and stores these parameters in a user preferences data structure.

### Step 5:

Server analyzes current input information together with preference parameters.

Server combines the newly received meal-related input with the existing preference parameters to form a comprehensive preference profile for the current request.

Input: Server uses the validated current input information and the preference parameters retrieved from the user_preferences structure.

Output: Server generates an enriched preference profile that includes explicit constraints (for example, no nuts, low sugar) and implicit tendencies (for example, strong preference for spicy meals at lunch), and stores it as a structured object for prompt generation.

### Step 6:

Server generates a prompt sentence for the generative AI model.

Server constructs a natural language text that encodes the enriched preference profile and operational constraints such as preparation time limits.

Input: Server uses the structured preference profile, including user taste preferences, dietary constraints, desired portion size, and target preparation window.

Output: Server outputs a prompt sentence, such as “Generate a dinner recipe for an adult that matches the following preferences: spicy taste, low sugar, no nuts, and suitable for preparation within 20 minutes. The recipe should list ingredients, quantities, and step-by-step cooking instructions.” Server stores this prompt sentence in memory and prepares it for tokenization.

### Step 7:

Server tokenizes the prompt sentence and prepares it for the generative AI model.

Server converts the text of the prompt sentence into token identifiers using a predefined vocabulary and applies any necessary preprocessing such as lowercasing or special token insertion.

Input: Server takes the prompt sentence in text form.

Output: Server produces a sequence of token identifiers representing the prompt, along with positional indices and optional control tokens, and passes this sequence into the generative AI model interface.

### Step 8:

Server invokes the generative AI model to generate recipe information.

Server feeds the token sequence into a neural network-based generative AI model and executes inference to produce an output token sequence representing recipe content.

Input: Server provides the prompt token sequence, model parameters, and decoding parameters such as beam width or temperature.

Output: Server computes a sequence of output tokens that represent the generated recipe, including ingredients, quantities, and cooking steps, and then converts the tokens back to text to obtain recipe information.

### Step 9:

Server parses the generated recipe information into structured data.

Server analyzes the generated text to extract ingredients, quantities, preparation steps, estimated cooking duration, and any implied constraints.

Input: Server uses the raw recipe text generated by the generative AI model.

Output: Server produces a structured recipe representation comprising lists of ingredients with normalized identifiers, numerical quantities, ordered step descriptions, and a computed preparation time estimate, stored in a recipe data structure.

### Step 10:

Server creates order information and associates it with the recipe.

Server instantiates a new order record linking the user identifier, the structured recipe, and requested pickup parameters.

Input: Server uses the structured recipe representation and the original validated input information including pickup location and pickup time.

Output: Server generates an order identifier, stores an order record in the database with initial status values, and associates references to recipe data and user data.

### Step 11:

Server generates cooking instruction information for the cooking facility.

Server transforms the structured recipe data into instructions that can be executed by the cooking facility, including item codes and target completion times.

Input: Server uses the structured recipe representation and computes the required start time by subtracting preparation duration from the requested pickup time.

Output: Server builds a cooking instruction message containing dish identifiers, ingredients, quantities, start and completion times, and any special handling notes, and transmits it to a cooking facility terminal.

### Step 12:

Cooking facility terminal displays and manages cooking tasks.

Terminal in the cooking facility receives the cooking instruction information from the server and presents it to kitchen staff.

Input: Terminal obtains the cooking instruction message including dish identifiers and schedule information.

Output: Terminal displays ordered tasks on a screen, allows staff to update statuses, and sends status updates such as “cooking started” or “cooking completed” back to the server as structured messages.

### Step 13:

Server receives cooking completion information and updates order state.

Server processes status messages from the cooking facility terminal indicating that preparation has been completed.

Input: Server receives a cooking completion message containing an order identifier and a completion timestamp.

Output: Server changes the corresponding order state in the database to indicate that the meal is ready for delivery, updates preparation time metrics, and stores the completion time for later delivery planning.

### Step 14:

Server generates delivery plan information.

Server uses the completion time, pickup location, and pickup time to compute an optimized delivery route and schedule.

Input: Server uses the order's pickup location coordinates, the requested pickup time, the actual completion time, and current delivery tasks for a delivery terminal.

Output: Server calculates estimated travel times, determines a route sequence and expected arrival times, produces delivery plan information, and stores it in the delivery_plans data structure before transmitting it to a delivery terminal.

### Step 15:

Delivery terminal receives delivery plan information and guides delivery.

Terminal used by delivery personnel obtains the delivery route and schedule from the server and uses positioning and mapping capabilities to guide movement.

Input: Terminal receives delivery plan information including route waypoints, sequence of stops, and planned arrival times.

Output: Terminal displays a map with navigation instructions, continuously updates its position using location services, and sends periodic progress reports back to the server, including current location and estimated arrival time deviations.

### Step 16:

Server monitors delivery progress and adjusts information for the user.

Server processes periodic location and status reports from the delivery terminal to maintain an accurate picture of delivery progress.

Input: Server receives coordinates, timestamps, and delivery status flags from the delivery terminal.

Output: Server updates internal delivery tracking records, recalculates estimated arrival times if necessary, and transmits updated delivery status information to the user terminal via notifications or API responses.

### Step 17:

Server generates authentication information for pickup at the storage apparatus.

Server produces a one-time authentication token that will be used by the user to unlock a specific compartment of the storage apparatus.

Input: Server uses the order identifier, associated storage apparatus identifier, and expected arrival window derived from the delivery plan.

Output: Server generates a secure token or numeric code, stores it in the pickup_tokens data structure with validity parameters, and sends the authentication information to the user terminal along with identifying information for the storage apparatus and compartment.

### Step 18:

User receives authentication information and prepares for pickup.

User operates the user terminal to view the code or token and confirms the pickup location and time.

Input: User terminal receives a message from the server containing the authentication code, storage apparatus identifier, and any instructions.

Output: User terminal displays the code in text form or as a representation suitable for manual entry, enabling the user to later input the code at the storage apparatus.

### Step 19:

User inputs authentication information at the storage apparatus.

User goes to the designated pickup location and operates the storage apparatus interface to provide the authentication information.

Input: User enters the received code using a keypad or selects a function on the storage apparatus interface to transmit the code.

Output: Storage apparatus transmits the entered authentication code, along with storage apparatus identifier and compartment identifier, to the server for validation.

### Step 20:

Server verifies authentication information and controls unlocking.

Server checks the received token against stored records and determines whether it should authorize unlocking of the storage apparatus.

Input: Server receives the entered code, storage apparatus identifier, and compartment identifier from the storage apparatus.

Output: Server validates the token by matching it to an unexpired, unused token associated with the same order and apparatus, then sends response information authorizing or denying unlock; upon authorization, the server updates the order state to indicate that pickup is in progress or completed.

### Step 21:

Storage apparatus unlocks the compartment and reports pickup completion.

Storage apparatus receives the server's response and actuates its lock mechanism accordingly.

Input: Storage apparatus obtains response information indicating whether to unlock a specific compartment.

Output: Storage apparatus energizes its lock actuator to unlock the compartment if authorized, detects door opening and closing events through sensors, and sends a pickup completion message to the server, allowing the server to finalize the order state as picked up.

### Step 22:

User optionally provides evaluation information after consumption.

User operates the user terminal after consuming the meal to rate the meal and submit feedback in text form.

Input: User enters a numerical rating and text comments describing preferences, satisfaction level, and any issues.

Output: User terminal sends a structured feedback payload to the server, which includes the order identifier, rating, and textual evaluation for future preference analysis and model updates.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented food recommendation and recipe-generation systems typically treat user preferences as static attributes and rely on simple rule-based filters or generic recommendation algorithms. Such systems merely map explicit user selections to pre-defined options and do not dynamically adapt underlying computational models in response to fine-grained user feedback or emotional state. As a result, these systems often generate prompt sentences for generative AI models in an ad hoc manner, without systematically encoding multi-modal user-related information, such as usage history, nutritional constraints, and emotion signals derived from sensor data. This leads to suboptimal utilization of the generative AI model, reduced prediction accuracy, and increased latency caused by repeated trial-and-error prompting on the server side.

Furthermore, existing architectures generally separate recommendation logic, resource allocation for preparation and delivery, and emotion recognition into siloed subsystems. The server therefore performs redundant data transformations, multiple database lookups, and disparate API calls for each subsystem, which increases computational overhead and complicates scaling. The lack of an integrated server-side control flow that jointly manages (i) acquisition and learning of user-related information, (ii) generation of optimized prompt sentences for a generative AI model, and (iii) orchestration of preparation, transport, and pickup resources results in inefficient use of processing resources and network bandwidth. In addition, many known systems do not incorporate multi-modal emotion estimation (from images and audio) as first-class computational inputs that directly influence how the server builds prompts and interprets generative AI outputs. As a consequence, these systems cannot automatically refine an internal, learned user model based on emotional reactions to generated outputs, and thus fail to improve the generative AI model's relevance over time. This deficiency manifests as a technical problem of how to structure server-side data processing, model updating, and prompt generation so that the generative AI model is driven by compact, information-rich prompt sentences representing an up-to-date user model, thereby improving computational efficiency and output quality in a systematic manner. Therefore, there is a need for a computer-implemented system and server functionality that: (i) unifies acquisition and storage of user-related information, including emotional state information derived from image and audio signals; (ii) programmatically generates, on the server, optimized prompt sentences for a generative AI model based on a learned user model; (iii) validates and filters the generative AI model outputs against constraints such as nutritional and resource conditions; and (iv) uses post-output feedback and emotion estimation results as learning data to iteratively update both the user-related information and the generative AI model. By solving these problems, it becomes possible to improve the operation of the server itself, reduce redundant processing, and produce higher-quality, context-appropriate food and beverage proposals with lower computational cost.

The specific processing by the specific processing unit 290 of the data processing device 12 in Application Example 2 is realized by the following means.

The present invention provides a server comprising a processor configured to receive input information from a user via an information input/output device, acquire, from a storage device, user-related information including the received input information, past usage history information of the user, and emotional state information, and analyze the user-related information to generate a prompt sentence including an instruction sentence for causing a generative artificial intelligence model to generate food-and-beverage provision content or cooking procedures based on preferences of the user; input the generated prompt sentence to the generative artificial intelligence model and acquire, from the generative artificial intelligence model, food-and-beverage provision candidate information based on the preferences and the emotional state of the user; determine, for each food-and-beverage provision candidate included in the food-and-beverage provision candidate information, conformity to nutritional conditions, restricted-component conditions, and provision conditions, and generate proposal information for the user by excluding or correcting candidates that do not conform; generate cooking instruction information for food-preparation equipment and transport instruction information for transport equipment based on the proposal information for the user, and transmit the cooking instruction information and the transport instruction information to the food-preparation equipment and the transport equipment; acquire vacancy-state information of a storage device for receiving goods or a handover facility, calculate a reception time slot and a reception location candidate corresponding to the food-and-beverage provision candidate information and present the reception time slot and the reception location candidate to the user, and update reservation information of the storage device for receiving goods or the handover facility based on a reception time slot and a reception location selected by the user; and acquire post-meal evaluation information and emotion estimation results via the information input/output device or via an image acquisition device and a sound acquisition device, and update the user-related information and the generative artificial intelligence model by using the post-meal evaluation information and the emotion estimation results as learning data. This enables the server to centrally control acquisition, analysis, and updating of user-related information and to automatically construct information-rich prompt sentences that drive the generative artificial intelligence model in a context-aware manner, thereby improving computational efficiency, reducing redundant processing, and enhancing the accuracy and personalization of generated food-and-beverage proposals in a computer-implemented environment.

The term “user” refers to an individual or group that interacts with the system via an information input/output device in order to provide preferences, evaluations, and other inputs and to receive proposals for food-and-beverage provision.

The term “processor” refers to one or more hardware processing units, such as central processing units or other computing circuitry, configured to execute computer-readable instructions to perform the functions described herein.

The term “storage device” refers to any non-transitory computer-readable medium, including but not limited to semiconductor memory, magnetic storage, or optical storage, that stores user-related information, models, prompt sentences, and other data used by the processor.

The term “information input/output device” refers to an apparatus, such as a terminal device, that presents information to the user and receives information from the user, including but not limited to a portable electronic device having a display, a touch interface, and communication functions.

The term “user-related information” refers to information associated with a particular user, including but not limited to input information provided by the user, past usage history information, preference information, evaluation information, and emotional state information derived from sensor data.

The term “preference information” refers to information indicating food-and-beverage preferences of the user, including but not limited to liked or disliked taste attributes, dietary restrictions, preferred ingredient types, and other selection tendencies.

The term “evaluation information” refers to information supplied by the user after provision of food-and-beverage content, including but not limited to ratings, textual comments, and structured responses to questionnaires that express satisfaction or dissatisfaction.

The term “past usage history information” refers to historical records of user interactions with the system, including but not limited to past orders, accepted proposals, rejected proposals, timing of usage, and context associated with such interactions.

The term “emotional state information” refers to data indicating an estimated emotional condition of the user, such as joy, neutrality, or displeasure, obtained from an emotion estimation model based on sensor inputs including images and audio.

The term “image acquisition device” refers to an apparatus capable of capturing image data of the user, such as a camera integrated in a terminal device or external imaging equipment, used to obtain facial expression images for emotion estimation.

The term “sound acquisition device” refers to an apparatus capable of capturing audio data of the user, such as a microphone included in a terminal device or external audio input equipment, used to obtain sound signals for emotion estimation.

The term “emotion estimation model” refers to a computational model that receives sensor data, including facial expression images or audio signals, and outputs estimated emotional state information such as emotion categories and associated confidence values.

The term “linguistic emotion analysis function” refers to a processing function that analyzes textual data derived from user speech or input and outputs estimated emotional states based on linguistic features.

The term “generative artificial intelligence model” refers to a model implemented by machine learning or other artificial intelligence techniques that receives a prompt sentence and generates content, such as food-and-beverage provision candidates or cooking procedures, in accordance with the prompt sentence.

The term “prompt sentence” refers to a text-based instruction sequence that encodes user-related information, constraints, and desired output format, and that is provided as input to the generative artificial intelligence model to control the generation of food-and-beverage provision content.

The term “food-and-beverage provision content” refers to information specifying a proposed food or beverage service, including but not limited to menu items, combinations of dishes, serving styles, and associated preparation details.

The term “cooking procedures” refers to information describing preparation steps for a food or beverage item, including but not limited to ingredient lists, quantities, processing steps, and timing instructions.

The term “food-and-beverage provision candidate information” refers to one or more candidate items generated by the generative artificial intelligence model, each candidate specifying food-and-beverage provision content potentially suitable for the user.

The term “nutritional conditions” refers to constraints or criteria related to nutritional properties of proposed food-and-beverage items, including but not limited to energy values, macronutrient ratios, and specific nutrient limits.

The term “restricted-component conditions” refers to constraints associated with components that should be limited or avoided in food-and-beverage items, including but not limited to allergens, prohibited ingredients, or components exceeding defined thresholds.

The term “provision conditions” refers to operational or contextual constraints under which food-and-beverage items are to be provided, including but not limited to time-of-day constraints, preparation capacity constraints, and delivery feasibility constraints.

The term “proposal information” refers to information generated by the processor based on food-and-beverage provision candidate information after applying constraints, the proposal information being prepared for presentation to the user as recommended options.

The term “food-preparation equipment” refers to one or more apparatuses or systems used to prepare food-and-beverage items, including but not limited to kitchen appliances, cooking lines, or automated preparation devices.

The term “transport equipment” refers to one or more apparatuses or systems used to transport food-and-beverage items from preparation locations to reception locations, including but not limited to delivery vehicles, robotic transport systems, and associated control systems.

The term “cooking instruction information” refers to data specifying operations to be executed by the food-preparation equipment, including but not limited to which items to prepare, how to prepare them, and by when they should be completed.

The term “transport instruction information” refers to data specifying operations to be executed by the transport equipment, including but not limited to pickup locations, destination locations, time windows, and handling requirements.

The term “storage device for receiving goods” refers to a storage apparatus configured to temporarily store prepared food-and-beverage items for later pickup by the user, including but not limited to lockers or other automated storage units.

The term “handover facility” refers to a facility configured to execute handover of food-and-beverage items to the user, including but not limited to pickup counters, kiosks, or other designated transfer points.

The term “vacancy-state information” refers to information indicating available capacity or time slots of the storage device for receiving goods or the handover facility, including identifiers of available compartments or resources and corresponding time intervals.

The term “reception time slot” refers to a time interval within which the user is permitted to receive a prepared food-and-beverage item from the storage device for receiving goods or the handover facility.

The term “reception location candidate” refers to one or more possible reception locations, including particular storage devices for receiving goods or handover facilities, presented to the user as options for receiving prepared food-and-beverage items.

The term “reservation information” refers to data that associates a user, an order, a reception time slot, and a reception location, and that is used by the storage device for receiving goods or the handover facility to manage allocation of resources.

The term “post-meal evaluation information” refers to evaluation information provided by the user after consumption of a food-and-beverage item, including ratings, comments, and structured assessment values reflecting satisfaction.

The term “emotion estimation result” refers to output data produced by an emotion estimation model or linguistic emotion analysis function indicating an estimated emotional state of the user at or after provision of a food-and-beverage item.

The term “learning data” refers to data used to train or update models within the system, including user-related information, post-meal evaluation information, and emotion estimation results, which are employed to refine user models or generative artificial intelligence models.

The term “preference profile” refers to an internal representation of the user's preferences, derived from usage history, evaluations, and emotional responses, and stored as part of the user-related information.

The term “learned user model” refers to a computational model, produced by machine learning or statistical analysis, that captures correlations between user-related information and user satisfaction, and that outputs feature information used to construct prompt sentences.

The term “feature information” refers to numerical or categorical descriptors derived from the learned user model that represent characteristics of the user's preferences, behavior, or emotional tendencies, and that are used in generating prompt sentences for the generative artificial intelligence model.

In one embodiment, a server cooperates with a terminal and a user to implement the claimed system. The server includes at least one hardware processor, a main memory, a non-transitory storage device, and a network interface. The terminal includes a display, an input interface such as a touch panel, a camera as an image acquisition device, and a microphone as a sound acquisition device. The server and the terminal communicate via a communication network using a secure transport protocol such as HTTPS with symmetric-key encryption such as AES.

The server executes an application program implemented, for example, using a server-side framework (such as a general-purpose web framework) and a data analysis environment using an interpreted language (such as Python), libraries for numerical computation (such as a numerical array library), data analysis (such as a data frame library), and machine learning (such as a machine learning library, a neural network framework, or a deep learning framework). The server additionally accesses a relational or non-relational database management system (such as a relational database engine or a key-value store) to persist user-related information, learned user models, prompt sentences, and generated candidate information.

The terminal executes an application implemented, for example, as a native mobile application using an operating system specific development environment. The terminal application provides user interface screens for receiving user preferences, collecting evaluation information, and displaying proposed food-and-beverage content. The terminal uses a camera access library (such as a standard camera API and a computer vision library) and a microphone access API to capture images and audio signals, which are then sent to the server as digital data streams.

The server stores user-related information in the storage device using a structured data model. In one example, the server stores a user profile table with fields such as a user identifier, aggregated preference attributes, and nutritional and restriction flags. The server stores an order history table with an order identifier, a user identifier, a list of item identifiers, timestamps, and contextual metadata. The server stores feedback and emotional state information in separate but relational tables, each row including an order identifier, a rating value, textual feedback, and one or more emotion labels derived from images and audio. The server normalizes these tables and maintains indexes for fast lookup by user identifier and order identifier.

The server implements an emotion estimation model as a trained neural network. For image-based emotion estimation, the server uses a convolutional neural network (CNN) comprising multiple convolution layers, pooling layers, and fully connected layers. The CNN receives as input a preprocessed face image extracted by the terminal or by the server using a computer vision library. The server normalizes pixel values, resizes the image to a fixed resolution, and applies data augmentation techniques such as random cropping and horizontal flipping during training. The CNN outputs a probability distribution over emotion classes such as joy, neutrality, sadness, or anger, using a softmax output layer. The server trains this CNN using a supervised learning method with a cross-entropy loss function, and updates network weights using a gradient descent-based optimizer such as stochastic gradient descent with momentum or Adam. The server periodically retrains the model with newly collected data, thereby adapting to user-specific expression patterns.

For audio-based emotion estimation, the server extracts acoustic features such as Mel-frequency cepstral coefficients (MFCCs), pitch contours, and energy statistics from recorded audio. The server segments the audio into fixed-length frames and computes, for each frame, a feature vector. The server then applies a recurrent neural network (RNN), such as a long short-term memory (LSTM) network or a gated recurrent unit (GRU) network, or a one-dimensional convolutional neural network, to these sequences of feature vectors. The audio emotion model outputs one or more emotion labels and confidence scores. The server combines the outputs from the image-based and audio-based emotion models using a fusion rule such as a weighted average or a shallow neural network that takes the emotion probabilities as input and outputs a fused emotional state.

The server builds a learned user model by integrating the user-related information stored in the database. In one embodiment, the server uses feature engineering to construct a feature vector for each historical order. The feature vector includes, for example, one-hot encoded representations of dish categories, continuous attributes such as standardized calorie and price values, and binary flags indicating presence of specific ingredients. The server additionally appends encoded preference information and emotional state information, such as a numerical representation of the dominant emotion and its confidence, to each feature vector. The server uses a machine learning algorithm such as gradient-boosted decision trees or a feed-forward neural network to predict a satisfaction score or a probability that the user will accept a candidate proposal. The server trains this model using historical orders as training instances, with labels derived from user ratings, textual sentiment analysis, and emotional responses. The server uses a loss function such as mean squared error or cross-entropy, and updates model parameters using gradient-based optimization.

The server maintains, for each user, a preference profile obtained by aggregating the output of the learned user model. This preference profile can include learned weights for various attributes such as cuisine type, spiciness, portion size, and time-of-day preference. The server periodically recomputes the preference profile by re-running the learning algorithm with the latest data, and stores the profile in a dedicated structure, such as a document-oriented record, for rapid retrieval during prompt sentence generation.

The server generates prompt sentences in natural language based on the user-related information and the learned user model. In one embodiment, the server executes a prompt generation module that receives, as input, the preference profile, the most recent emotional state labels, and the current context such as date, time, and resource availability. The prompt generation module applies a rule-based template system that selects a template sentence pattern and inserts variable phrases representing user preferences and constraints. The server uses this rule-based approach to ensure that the prompt sentences consistently encode sufficient technical constraints for the generative AI model.

For example, the server may generate the following prompt sentence:

“User 123 is vegetarian, strongly prefers spicy dishes, and recently showed joy when eating a spicy pasta. Propose three dinner menus with descriptions and approximate calories. Exclude any dishes containing nuts. Return the result as a list with each item having a name, a description, and a calorie estimate.”

In another example, the server may generate the following prompt sentence:

“The user prefers spicy vegetarian dishes and cannot eat nuts. Generate three dinner menu options suitable for a weekday evening, with descriptions and calorie estimates.”

The server then transmits the prompt sentence to an external or internal generative AI model. In one embodiment, the generative AI model is a large language model implemented as a multi-layer transformer neural network. The transformer includes an encoder-decoder architecture or a decoder-only architecture with self-attention mechanisms. The server sends the prompt sentence over an application programming interface, specifying model parameters such as a maximum token length and a sampling temperature. The generative AI model processes the prompt sentence by tokenizing it, computing contextual embeddings through multiple transformer blocks, and then generating tokens representing menu and recipe text according to its learned probability distribution.

The server receives the generated text output from the generative AI model. The server uses pattern-matching routines and a parser to extract structured fields such as menu names, descriptions, and calorie estimates from the generated text. The server then applies validation logic to this structured data. This logic includes checking each candidate for violation of restricted-component conditions such as allergens and banned ingredients, verifying that calorie estimates fall within a specified range derived from nutritional conditions, and ensuring that preparation time is compatible with current food-preparation equipment capacity. If a candidate fails any constraint, the server either discards the candidate or modifies it according to predetermined adjustment rules. For example, the server may remove an ingredient from an ingredient list if that ingredient appears in a stored list of restricted components for the user.

The server generates proposal information as a set of validated food-and-beverage candidates. The server ranks the candidates based on the output of the learned user model, which predicts a satisfaction score for each candidate given its feature representation. The server selects the top-ranked candidates and sends them to the terminal for display.

The server also controls physical food-preparation equipment and transport equipment. The server translates selected candidates into cooking instruction information that includes identifiers of recipes, required ingredients, cooking temperatures and times, and target completion times. The server sends this information to the food-preparation equipment control system over a network, using a structured messaging protocol. The food-preparation equipment may include programmable ovens, cookers, and automated dispensers that interpret the instruction information to execute precise control sequences. The server thereby causes the equipment to operate according to technical parameters derived from the computational outputs.

The server similarly generates transport instruction information for transport equipment, such as delivery vehicles or robotic carriers. The server computes optimal routes using a routing algorithm and prepares an instruction message specifying pickup locations, destination locations, and delivery time windows. The server sends this message to a transport management system, which controls the transport equipment to fulfill the delivery. By coordinating these instructions, the server ensures that the physical preparation and delivery processes are aligned with the computational recommendations generated by the generative AI model and constrained by the learned user model.

The server further manages storage devices for receiving goods and handover facilities such as lockers or pickup counters. The server periodically retrieves vacancy-state information from these devices through their respective control systems. The vacancy-state information includes, for example, identifiers of available compartments and associated time slots. The server stores this information in the database and queries it when generating proposals, thus enabling the server to present feasible reception time slots and reception location candidates within the terminal application.

The terminal displays to the user the proposed reception time slots and locations. The user selects a desired slot and location. The terminal sends this selection to the server, which updates reservation information in the database and transmits reservation commands to the storage device or handover facility. The storage device responds by reserving a compartment and associating it with the order, thereby enabling automatic access control. The server generates access tokens, which may be encoded in a QR code displayed on the terminal. The storage device later verifies these tokens with the server to permit controlled retrieval of the prepared food-and-beverage items.

After the user receives and consumes the items, the terminal presents feedback screens and optionally initiates emotion capture. The user enters post-meal evaluation information such as ratings and comments, and the terminal sends this information to the server. The terminal also captures images and audio, and either processes them locally or forwards them for server-side emotion estimation using the models described earlier. The server stores post-meal evaluation information and emotion estimation results in the database and uses them as learning data to update the learned user model and, in certain embodiments, to fine-tune the generative AI model.

The server thereby implements a feedback loop: the server uses accumulated learning data to retrain or fine-tune models on the storage device. During training, the server constructs mini-batches of feature vectors and labels, computes forward passes through the model, calculates a loss value such as cross-entropy or mean squared error, and performs backpropagation to update network weights. The server may also use techniques such as regularization, early stopping, and learning rate schedules to improve convergence and prevent overfitting. As a result, prediction accuracy for satisfaction scores increases, and the generative AI model is provided with more informative prompt sentences.

The described arrangement improves computer technology in several respects. First, the server reduces communication and processing overhead by generating compact, information-dense prompt sentences rather than repeatedly querying the generative AI model with incomplete or naive prompts. This optimization reduces the number of network round-trips and the computational load on the generative AI model. Second, the server structures user-related information into specialized data structures and uses them to compute feature vectors for machine learning, which allows the system to efficiently retrieve and process information with reduced latency and improved cache performance. Third, the modular architecture of the emotion estimation model, the learned user model, and the prompt generation module enables the server to adjust each component independently while preserving overall data flow consistency, thereby improving maintainability and scalability.

Moreover, the server does not simply automate human decision-making but executes non-traditional computational processes that are impractical for a human to perform manually in real time. For example, the server jointly optimizes user satisfaction, nutritional constraints, resource availability, and physical device control by simultaneously processing large quantities of historical data, multi-modal sensor inputs, and system-state information. The server uses field-specific neural architectures (such as CNNs for images and LSTMs for audio) and structured feature representations to derive patterns from emotional responses that would be difficult for a human to formalize reliably. These patterns are then injected into prompt sentences and into candidate evaluation logic, resulting in a non-conventional integration of generative AI output and device control.

The causal relationship between these technical arrangements and the technical effects can be summarized as follows. By fusing emotion estimation outputs with user preference profiles, the server learns a more precise mapping from candidate features to predicted satisfaction, which reduces error in acceptance prediction and thus lowers the number of rejected recommendations and re-queries to the generative AI model. This directly lowers processing time and network usage. By validating and correcting generative AI outputs using algorithmic constraints on the server before transmission to preparation and transport equipment, the system prevents invalid instructions from reaching devices, thereby reducing failure rates and avoiding costly retries. The combined effect is improved computational efficiency, enhanced accuracy of recommendations, and more stable operation of the overall computer-controlled food-and-beverage provisioning system.

In alternative embodiments, the server may embed the generative AI model locally rather than accessing it as a remote service. In such a case, the server loads model parameters from the storage device into memory and executes the transformer model directly using optimized linear algebra libraries. The server can then adapt the model incrementally using user-specific data, adjusting a subset of parameters such as adapter layers or embedding vectors while freezing the core network. In another variation, the terminal may perform some or all of the emotion estimation processing locally using a lightweight neural network, thereby reducing uplink communication of raw image and audio data and further lowering network load. In yet another embodiment, the server may use a different class of generative AI model, such as a sequence-to-sequence model tailored for recipe generation, but still receives a prompt sentence constructed according to the mechanisms described above. The server may also vary the algorithm used to generate ranking scores for candidate menus, for example, by combining predicted satisfaction probability with a diversity measure so that the proposals are not only accurate but also varied. Such variations still fall within the scope of the claimed system, as they retain the essential features of integrating user-related information, including multi-modal emotional state information, with prompt sentence generation for a generative AI model and coordinated control of preparation, transport, and reception equipment.

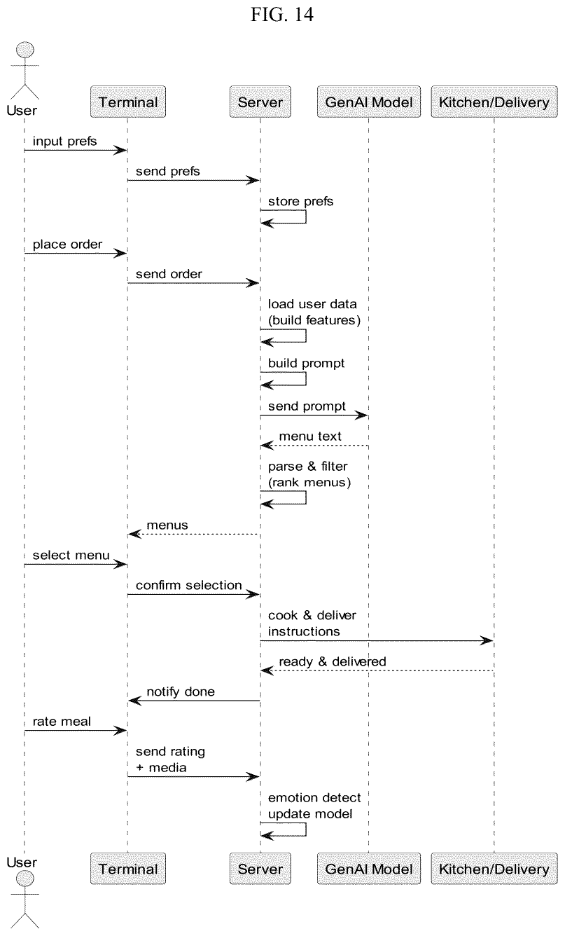

The following describes the processing flow using FIG. 14.

### Step 1:

User operates the terminal to register initial preferences.

User opens an application on the terminal, selects preferred attributes such as taste (for example, spicy, mild), dietary style (for example, vegetarian), and restricted components (for example, allergens), and confirms the input.

Input: raw preference selections from user interface elements (checkboxes, sliders, text fields).

Output: a structured preference object in the terminal memory (for example, a key-value map).

Terminal converts the user selections into a structured data object, validates required fields, and stores it temporarily for transmission.

### Step 2:

Terminal sends the preference object to the server.

Terminal serializes the preference object into a JSON document and attaches a user identifier and device identifier, then opens a secure HTTPS connection and transmits the document to an API endpoint on the server.

Input: in-memory preference object and user identifier.

Output: encrypted HTTP request payload delivered to the server.

Terminal applies an encryption protocol (for example, TLS with AES) at the transport layer so that the payload is protected during transmission.

### Step 3:

Server receives and stores the preference data.

Server accepts the HTTPS request, decrypts the payload at the network stack, and passes the JSON body to an application handler.

Input: JSON document including user identifier and preference attributes.

Output: one or more database records in a user profile table.

Server parses the JSON into an internal data structure, performs schema validation, converts symbolic preference values into normalized codes, and executes a database insert or update query so that the preferences are persisted with indexes on the user identifier.

### Step 4:

User uses the terminal to place an order.

User browses menu options displayed on the terminal, selects one or more items (for example, a main dish and a side dish), optionally selects a desired time window, and confirms the order.

Input: user interactions on the menu selection screen.

Output: an order object in the terminal memory containing item identifiers, quantities, and requested time information.

Terminal aggregates the selections into a list structure, associates it with the user identifier, and prepares it for submission to the server.

### Step 5:

Terminal submits the order to the server.

Terminal packages the order object in a JSON document and sends it via HTTPS to an order-receiving endpoint on the server.

Input: order object with item identifiers, quantities, and user identifier.

Output: encrypted HTTP request payload containing order data.

Terminal ensures that a unique client-side order identifier and timestamp are added before transmission.

### Step 6:

Server records the order and retrieves user-related information.

Server receives the order JSON, parses it, and writes an order record into an orders table with fields such as order identifier, user identifier, item list, and status.

Input: parsed order object from the HTTP request.

Output: a persistent order record and a combined user-related information structure.

Server then queries the user profile table, order history table, feedback table, and emotion table by user identifier, joins the results, and constructs a user-related information structure that includes preferences, historical ratings, and recent emotional state labels.

### Step 7:

Server computes feature vectors from user-related information.

Server converts symbolic attributes (such as cuisine type and taste attributes) and emotional labels into numerical feature vectors.

Input: user-related information object containing preferences, history, and emotions.

Output: one or more numerical feature vectors representing the user state and context.

Server applies one-hot encoding to categorical fields, scales continuous values (such as calories and price) using normalization, and appends numerical codes for recent emotion categories and recency weights, producing a fixed-length feature vector that can be processed by machine learning models.

### Step 8:

Server updates or evaluates a learned user model.

Server loads model parameters of a learned user model from storage into memory and feeds the feature vectors into the model to compute predicted satisfaction scores for various attribute combinations.

Input: feature vectors representing a particular user and context.

Output: predicted preference parameters (for example, weights for spiciness, dish types, and serving sizes).

Server performs matrix multiplications, non-linear activations, and, if in training mode, gradient computations using a configured loss function, then updates the model weights and stores updated parameters back to the storage device.

### Step 9:

Server constructs a prompt sentence for the generative AI model.

Server uses the predicted preference parameters and current order context to fill a natural-language template.

Input: user profile values, predicted preference parameters, and contextual information such as mealtime and restrictions.

Output: a text string representing a prompt sentence.

Server selects a template pattern and inserts variable segments describing the user's preferences, restrictions, desired number of suggestions, and required output format, for example:

“User 123 is vegetarian, strongly prefers spicy dishes, and recently showed joy when eating a spicy pasta. Propose three dinner menus with descriptions and approximate calories. Exclude any dishes containing nuts. Return the result as a list with each item having a name, a description, and a calorie estimate.”

### Step 10:

Server sends the prompt sentence to the generative AI model.

Server prepares a request message that includes the prompt sentence, maximum token length, and sampling parameters, and sends this message to an interface of a generative AI model.

Input: prompt sentence string and model control parameters.

Output: a generated text output received from the generative AI model.

Server transmits the request via a model API, waits for completion, and receives the full generated response string in a buffered stream.

### Step 11:

Server parses the generative AI model output into candidate structures.

Server scans the generated text and extracts structured information for each proposed menu item.

Input: raw text response from the generative AI model.

Output: a list of candidate menu structures, each with fields such as name, description, and calorie estimate.

Server uses delimiters or pattern rules to identify item boundaries, applies regular expressions to capture labeled fields, converts numeric substrings to numeric types, and builds an internal array or list of candidate objects.

### Step 12:

Server validates candidates against constraints.

Server evaluates each candidate against nutritional conditions, restricted-component conditions, and operational provisions.

Input: list of candidate menu structures and constraint data (nutritional rules, restricted ingredient lists, preparation capacity data).

Output: a filtered and possibly modified list of valid candidate menus.

Server checks each candidate's ingredients and calorie estimate against stored rules; if an ingredient is on a restriction list, the server marks the candidate as invalid or removes that ingredient according to predefined replacement rules. The server also verifies that estimated preparation time fits within available equipment capacity windows; candidates that fail constraints are discarded.

### Step 13:

Server ranks valid candidates using the learned user model.

Server represents each valid candidate as a feature vector and passes it through the learned user model to obtain a predicted satisfaction score.

Input: valid candidate menu structures and user feature representation.

Output: a sorted list of candidates ordered by predicted satisfaction.

Server computes model outputs for each candidate, then performs a sorting operation based on the predicted scores, and selects a top subset (for example, top three) for presentation.

### Step 14:

Server sends ranked candidate menus to the terminal.

Server converts the selected candidate menus into a compact response document and returns it to the terminal via HTTPS.

Input: top-ranked candidate menu list.

Output: HTTP response payload containing candidate menu details for display.

Server may include additional metadata such as ranking scores and reason codes (for example, “recommended because you liked spicy pasta”) in the response.

### Step 15:

Terminal displays the candidate menus and receives user selection.

Terminal processes the response, populates a list view, and renders each candidate's name and description on the display.

Input: candidate menu list from the server.

Output: final selected candidate identifier and any user-specified customizations.

User taps one of the displayed candidates and optionally adjusts options (for example, removing a side dish), and the terminal updates an internal order structure, then confirms the selection.

### Step 16:

Terminal submits the final candidate selection to the server.

Terminal sends a confirmation message including the chosen candidate identifier, selected options, and user identifier.

Input: final candidate menu selection object.

Output: confirmation JSON payload delivered to the server.

Terminal uses HTTPS to transmit this payload, ensuring integrity and confidentiality.

### Step 17:

Server generates cooking instruction information.

Server maps the chosen candidate menu to one or more recipe identifiers and technical parameters required by food-preparation equipment.

Input: final candidate menu structure and equipment capability data.

Output: a cooking instruction object specifying operations for the equipment.

Server retrieves recipe definitions from a recipe table, computes timing and sequencing information based on current load and target completion time, and constructs a machine-readable instruction sequence (for example, a list of steps with temperatures and durations) for the preparation system.

### Step 18:

Server generates transport instruction information.

Server determines the pickup or delivery destination and time window and plans a route for transport equipment.

Input: order destination, reception time slot, and candidate menu details.

Output: a transport instruction object containing pickup and drop-off details.

Server consults the vacancy-state information of storage devices or handover facilities, selects a feasible reception location, and then uses a routing algorithm to set waypoints and schedule. The server encodes this information in a structured format that a transport management module can interpret.

### Step 19:

Server transmits cooking and transport instructions to external systems.

Server sends the cooking instruction object to a kitchen control system and the transport instruction object to a transport control system over network interfaces.

Input: cooking instruction object and transport instruction object.

Output: network messages that cause external systems to schedule and execute tasks.

Server updates the internal order status to indicate that preparation and transport have been scheduled and logs identifiers returned by the external systems for correlation.

### Step 20:

Server manages reservation of storage devices or handover facilities.

Server calculates feasible reception time slots and locations from vacancy-state information and prior order commitments.

Input: vacancy-state information and the order's expected ready time.

Output: a reserved slot record in reservation storage and an associated access token.

Server executes an algorithm that selects a slot that satisfies both user preferences and system constraints, writes a reservation record to a database, and generates a unique token (for example, a random string) that will later be encoded into a QR code.

### Step 21:

Terminal displays reception time and access information to the user.

Terminal receives from the server the reserved time slot, location, and an access token.

Input: reservation confirmation payload with slot information and token.

Output: user-visible details and an internally stored token for later QR-code generation.

Terminal renders the time and location on a confirmation screen and stores the token in local secure storage.

### Step 22:

Terminal generates and displays a QR code at pickup time.

Terminal, when requested by the user, uses the stored token to generate a QR code image.

Input: access token string from secure storage.

Output: pixel buffer representing a QR code displayed on the screen.

Terminal applies a QR-code generation algorithm that encodes the token into binary patterns, draws the pattern on a bitmap canvas, and displays the resulting image in full-screen mode for scanning.

### Step 23:

Server verifies the QR token and authorizes access.

Server receives a verification request from a storage device or handover facility containing the token read from the QR code.

Input: access token from external equipment.

Output: an authorization decision and associated compartment or handover information.

Server searches the reservation storage for a matching, unused token, checks that the current time falls within the reserved time slot, and returns an authorization response with a flag indicating permission and an identifier of the reserved compartment. If verification fails, the server returns a denial code.

### Step 24:

User retrieves the prepared item and completes the order.

User opens the compartment or receives the item from the handover facility once access is granted.

Input: physical access granted by the storage device or handover facility.

Output: completion of retrieval and a changed order status in the server records.

Storage device or handover facility notifies the server that retrieval has occurred, and the server updates the order status to “completed” in the database.

### Step 25:

Terminal collects post-meal evaluation information from the user.

Terminal prompts the user to rate the meal and provide comments and displays UI controls such as rating stars and text fields.

Input: user's rating input and textual feedback.

Output: a feedback object containing structured evaluation values and free-form text.

Terminal stores the feedback temporarily in memory until submission.

### Step 26:

Terminal sends feedback and optional sensor data to the server.

Terminal packages the feedback object and, if consent is given, metadata for captured images and audio, and transmits them via HTTPS to the server.

Input: feedback object and identifiers of associated sensor data.

Output: encrypted feedback payload delivered to the server and, optionally, raw or preprocessed sensor data.

Terminal may precompress images or audio to reduce data size before transmission.

### Step 27:

Server stores feedback and performs emotion estimation if needed.

Server receives the feedback payload, stores the evaluation fields in a feedback table, and uses associated image and audio data to infer an emotional state.

Input: feedback data and sensor data references or content.

Output: database records for feedback and emotion estimation results.

Server passes image data through the CNN to obtain emotion probabilities, passes audio features through the audio emotion model, fuses the outputs, and writes the resulting emotion labels and confidence values into an emotion table linked to the order.

### Step 28:

Server updates the learned user model and user profile.

Server retrieves new feedback and emotion data and combines them with the existing training set.

Input: accumulated training instances from orders, feedback, and emotions.

Output: updated model parameters and an updated preference profile for the user.

Server builds mini-batches of feature vectors and labels, re-runs the training procedure for the learned user model, updates the model weights using gradient-based optimization, recalculates aggregated preference weights (for example, spiciness preference strength), and stores the updated values in the user profile record.

### Step 29:

Server refines future prompt sentences based on updated knowledge.

Server modifies template selection rules and value ranges for inserted phrases based on the new preference profile and emotion patterns.

Input: updated preference profile and emotion statistics.

Output: future prompt sentences that more accurately reflect the user's current tastes and emotional reactions.

Server adjusts how often certain attributes (for example, spiciness or portion size) are explicitly mentioned in prompt sentences and may introduce new constraint phrases (for example, “avoid overly large portions”) derived from learned patterns, thereby increasing the precision and efficiency of subsequent interactions with the generative AI model.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

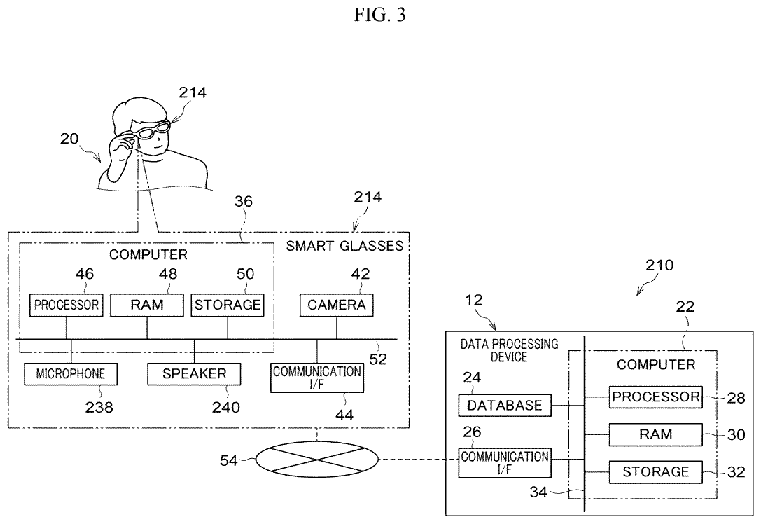

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

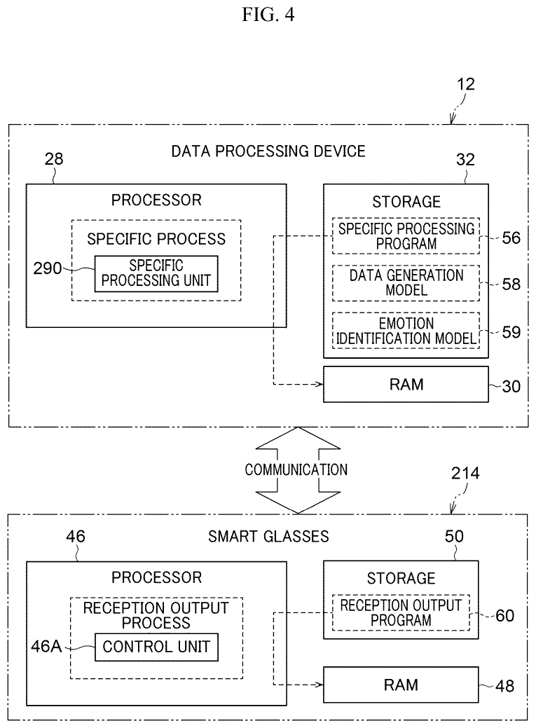

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

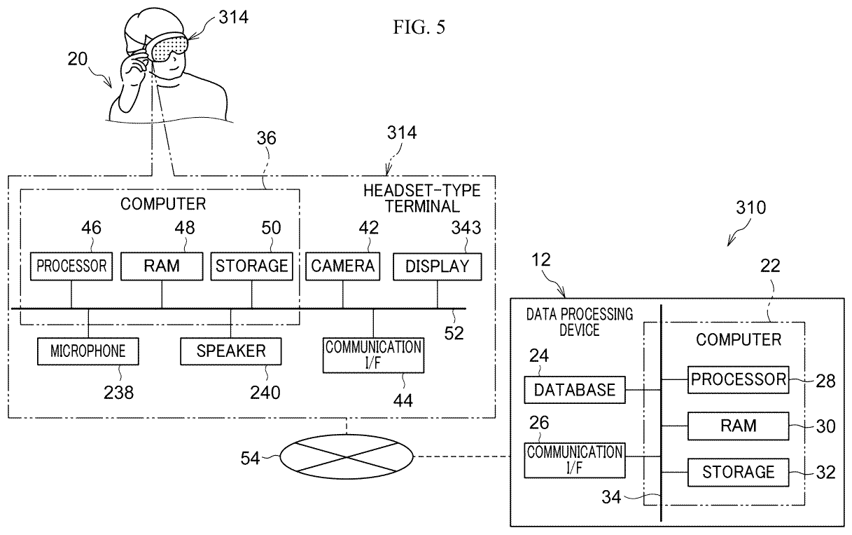

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

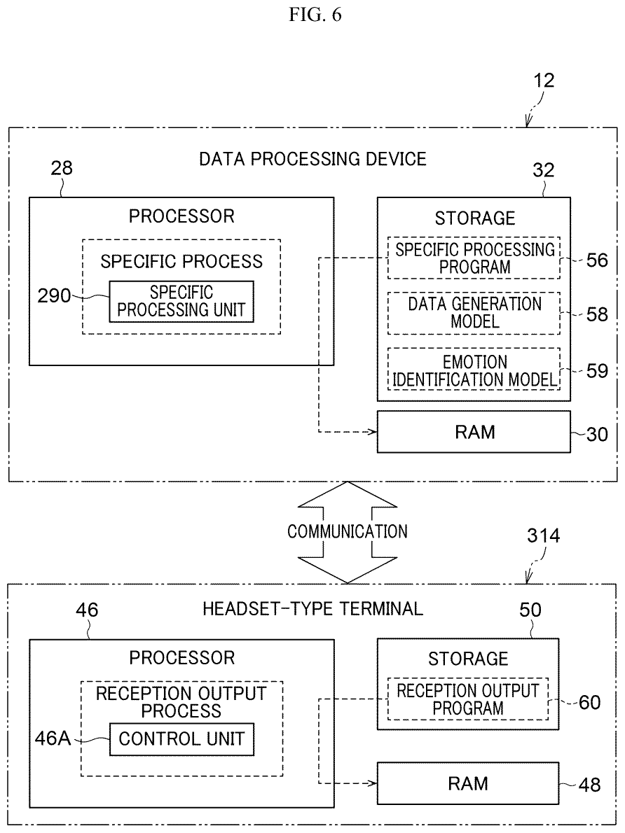

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network.

The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

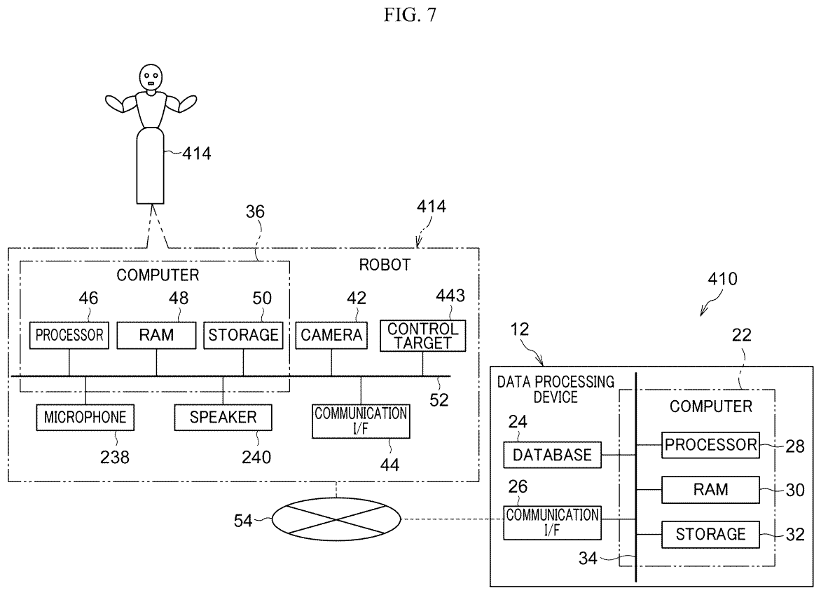

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

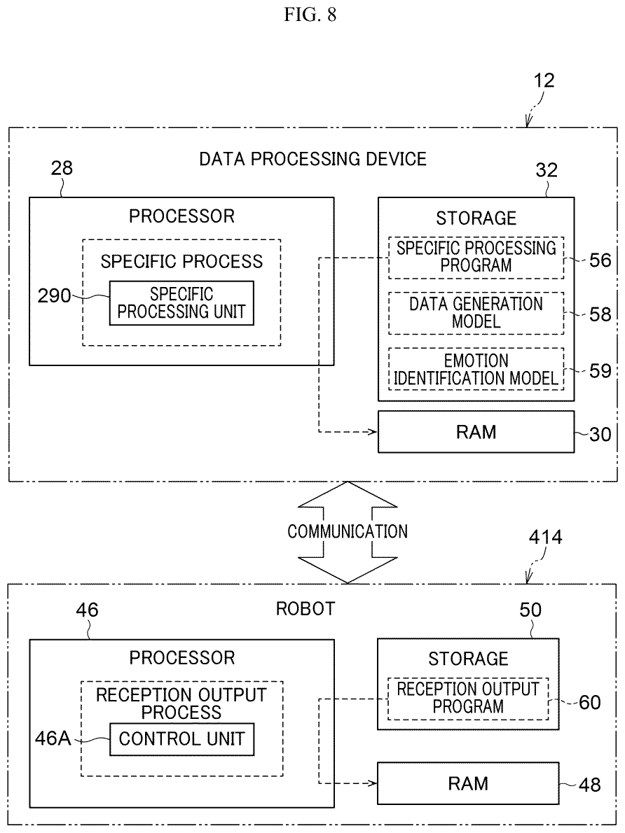

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network.

The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

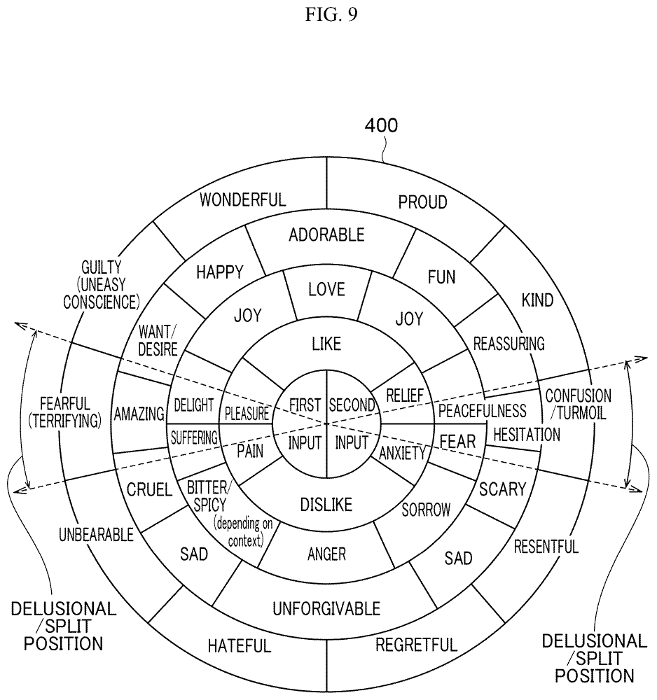

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles.

Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University).

Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

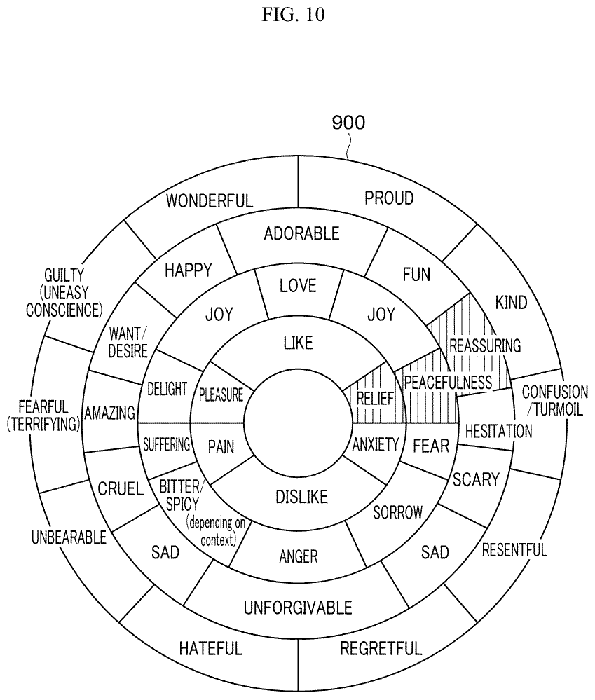

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
receive preference information and feedback information regarding food from a user via an information processing terminal,
acquire the preference information and past feedback information of the user from a storage unit, and generate, based on the acquired information, a prompt sentence in a natural language to be input to a generative AI model,
input the generated prompt sentence to the generative AI model and cause the generative AI model to generate recipe information including cooking procedure information and ingredient information corresponding to a preference of the user,
generate cooking instruction information indicating contents of cooking work to be performed by a cooking apparatus based on the generated recipe information, and transmit the cooking instruction information to an information processing terminal installed in a cooking facility,
execute route optimization processing to optimize a delivery route based on the recipe information and location information of a delivery destination, and transmit delivery instruction information based on a result of the route optimization processing to a delivery information processing terminal, and
acquire feedback information including an evaluation of the recipe information and a delivery from the user, analyze the feedback information by using a machine learning algorithm to update a parameter representing the preference of the user, and reflect the updated parameter in generation of a subsequent prompt sentence.

### (Supplementary 2)

The system according to supplementary 1,
wherein the processor is configured to
generate the prompt sentence by integrating the preference information of the user and the preference parameter updated by the machine learning algorithm into the prompt sentence including a natural language expression and an output format specification such that structured recipe information is obtained from the generative AI model.

### (Supplementary 3)

The system according to supplementary 1,
wherein the processor is configured to
convert the location information of the delivery destination into position information, determine a delivery order and a movement route for a plurality of delivery destinations by performing a calculation based on time information and distance information, and provide the determined delivery order and movement route as the delivery instruction information to the delivery information processing terminal.

### Application Example 1

### (Supplementary 1)

A system comprising a processor,
wherein the processor is configured to
provide a user interface through an information processing terminal to receive preference information and feedback information related to meals from a user, and to acquire the preference information and the feedback information from the information processing terminal,
analyze the acquired preference information and the acquired feedback information as user information to extract meal constraint conditions, flavor tendencies, and ingredient preferences of the user, and generate a prompt sentence to be input to a generative AI model in accordance with an extraction result,
input the generated prompt sentence and candidate menu information associated with the user information to the generative AI model, and cause the generative AI model to generate recipe information or menu description information suitable for preferences of the user,
generate and transmit cooking instruction information including cooking target items and cooking conditions to a cooking facility based on the recipe information or the menu description information, and generate and transmit delivery instruction information including a delivery route and an estimated arrival time to a delivery facility,
update a delivery status based on position information and delivery state information acquired from the delivery facility, and notify the updated delivery status to the information processing terminal,
input the feedback information and history information related to the delivery status to a machine learning model to cause the machine learning model to learn preference patterns and evaluation tendencies for each user, and update processing for generating the prompt sentence and processing for selecting the candidate menu information based on a learning result, and
update and store user profile information used for generation of recipe information or menu description information in subsequent operations based on the learning result and the user information.

### (Supplementary 2)

The system according to supplementary 1,
wherein the processor is configured to
analyze the prompt sentence by natural language processing to extract semantic information related to taste orientation, nutritional constraints, and cooking conditions of the user from the prompt sentence, and convert the extracted semantic information into an input format for the generative AI model to cause the generative AI model to generate the recipe information or the menu description information suitable for the preferences of the user.

### (Supplementary 3)

The system according to supplementary 1,
wherein the processor is configured to
acquire, as the feedback information, evaluation value information of the user, free-text information, and order history information, execute feature extraction processing based on natural language processing on the free-text information to generate satisfaction indices for attributes including spiciness level, salt level, and fat content, and cause the machine learning model to learn the satisfaction indices so as to sequentially update the preferences of the user reflected in the prompt sentence and the candidate menu information.

### Example 2

### (Supplementary 1)

A system comprising a processor,
wherein the processor is configured to
provide, on an information processing device, an interface for receiving input information related to meals from a user,
analyze, on the information processing device, the input information and past usage history information and evaluation information of the user to derive preference information of the user regarding meals, and generate a prompt sentence for instructing a generative AI model to generate a recipe or meal menu corresponding to the preference information, input the prompt sentence to the generative AI model and acquire recipe information based on the preference information of the user,
output, based on the recipe information, cooking instruction information to a cooking facility, the cooking instruction information including meal content to be prepared and a preparation completion time,
generate delivery plan information based on cooking completion information from the cooking facility and pickup location information and pickup time information specified by the user, and output the delivery plan information to a mobile terminal so as to determine a delivery route and delivery time,
generate authentication information used for receiving the meal stored in a storage apparatus installed at the pickup location, transmit the authentication information to a user terminal, and store the authentication information in association with the storage apparatus, and
determine validity of an input result of the authentication information transmitted from the storage apparatus, transmit response information for controlling unlocking of the storage apparatus to the storage apparatus when the authentication information is valid, and update a state of corresponding order information.

### (Supplementary 2)

The system according to supplementary 1,
wherein the processor is configured to
analyze the prompt sentence and text data of the evaluation information of the user by using a natural language processing technique, extract feature quantities representing the preference information of the user, and adjust input content to the generative AI model.

### (Supplementary 3)

The system according to supplementary 1,
wherein the processor is configured to periodically execute a learning process to learn preference parameters for each user based on the past usage history information and the evaluation information of the user, and generate the prompt sentence, the cooking instruction information, and the delivery plan information by using the updated preference parameters.

### Application Example 2

### (Supplementary 1)

A system comprising a processor,
wherein the processor is configured to
receive input information from a user via an information input/output device for acquiring preference information and evaluation information from the user; and
acquire user-related information including the received input information, past usage history information of the user, and emotional state information from a storage device, analyze the user-related information, and generate a prompt sentence including an instruction sentence for causing a generative artificial intelligence model to generate food-and-beverage provision content or cooking procedures based on preferences of the user; and
input the generated prompt sentence to the generative artificial intelligence model and acquire, from the generative artificial intelligence model, food-and-beverage provision candidate information based on the preferences and the emotional state of the user; and
determine, for each food-and-beverage provision candidate included in the food-and-beverage provision candidate information, conformity to nutritional conditions, restricted-component conditions, and provision conditions, and generate proposal information for the user by excluding or correcting candidates that do not conform; and
generate cooking instruction information for food-preparation equipment and transport instruction information for transport equipment based on the proposal information for the user, and transmit the cooking instruction information and the transport instruction information to the food-preparation equipment and the transport equipment; and
acquire vacancy-state information of a storage device for receiving goods or a handover facility, calculate a reception time slot and a reception location candidate corresponding to the food-and-beverage provision candidate information and present the reception time slot and the reception location candidate to the user, and update reservation information of the storage device for receiving goods or the handover facility based on a reception time slot and a reception location selected by the user; and
acquire post-meal evaluation information and emotion estimation results from the user via the information input/output device or via an image acquisition device and a sound acquisition device, and update the user-related information and the generative artificial intelligence model by using the post-meal evaluation information and the emotion estimation results as learning data.

### (Supplementary 2)

The system according to supplementary 1,
wherein the processor is configured to
use, as the emotional state information included in the user-related information, an output result of an emotion estimation model that performs emotion estimation processing based on a facial expression image acquired by the image acquisition device and an output result of an emotion estimation model or a linguistic emotion analysis function that performs emotion estimation processing based on a sound signal acquired by the sound acquisition device, and reflect the emotional state information in the prompt sentence so as to acquire, from the generative artificial intelligence model, the food-and-beverage provision candidate information corresponding to the emotional state.

### (Supplementary 3)

The system according to supplementary 1,
wherein the processor is configured to
use, in generation of the prompt sentence, a learned user model obtained by integrating, for each user, an updated preference profile, an order history after a previous provision, post-meal evaluation information, and emotion estimation results, and embed, in the prompt sentence, natural language information or structured information including feature information obtained from the learned user model, thereby individually optimizing, for each user, the food-and-beverage provision candidate information output from the generative artificial intelligence model.

What is claimed is:

1. A system comprising:
circuitry configured to:
receive, via a communication interface coupled to a packet-switched network, preference data and feedback data from a terminal device;
retrieve, from a storage device, historical preference data associated with the terminal device;
analyze the preference data, the feedback data, and the historical preference data to generate constraint parameters, and construct a prompt data structure including the constraint parameters and an instruction sequence directing a generative neural network model to generate structured output data conforming to the constraint parameters in a predetermined output format;
transmit the prompt data structure to the generative neural network model and receive response data including the structured output data comprising procedure data and component data; and
generate instruction data on the basis of the structured output data and transmit the instruction data to a second device via the packet-switched network.

2. The system according to claim 1, wherein the circuitry analyzes the preference data by applying a natural language processing operation that extracts constraint tokens from the preference data and classifies each constraint token into at least one of a restriction category, a affinity category, and a quantity category.

3. The system according to claim 2, wherein the circuitry further analyzes the feedback data by extracting evaluation scores and free-text comments, maps the evaluation scores to adjustment vectors in a preference feature space, and updates the historical preference data stored in the storage device on the basis of the adjustment vectors.

4. The system according to claim 3, wherein the restriction category includes dietary restriction data and allergen exclusion data, the affinity category includes flavor tendency data and ingredient preference data, and the quantity category includes portion size data, and the circuitry generates the constraint parameters by combining the classified constraint tokens with the updated historical preference data.

5. The system according to claim 4, wherein the structured output data represents recipe information including cooking procedure steps and ingredient lists, and the circuitry constructs the prompt data structure to include candidate menu information associated with the constraint parameters and to instruct the generative neural network model to generate the recipe information matching meal preferences of a user of the terminal device.

6. The system according to claim 1, wherein the generative neural network model comprises a transformer architecture including a token embedding layer, a positional encoding mechanism, a plurality of self-attention layers, and feedforward sublayers, and the predetermined output format specifies delimited fields for the procedure data and the component data.

7. The system according to claim 6, wherein the circuitry parses the response data by identifying field delimiters in the predetermined output format, extracts the procedure data as an ordered sequence of operation steps, and extracts the component data as a list of items each associated with a quantity value and a unit value.

8. The system according to claim 7, wherein the circuitry evaluates the structured output data against the constraint parameters by verifying that each item in the component data does not match any entry in the restriction category of the constraint parameters, and when a match is detected, constructs a revised prompt data structure requesting the generative neural network model to substitute the matched item with a compliant alternative.

9. The system according to claim 1, wherein the second device is a control terminal installed at a preparation facility, and the instruction data includes target item identifiers derived from the component data and operation parameters derived from the procedure data.

10. The system according to claim 9, wherein the operation parameters include a preparation sequence, a temperature setting, a duration value, and a completion time, and the circuitry generates the instruction data by mapping each operation step in the procedure data to a corresponding control command interpretable by equipment at the preparation facility.

11. The system according to claim 10, wherein the circuitry receives, from the second device via the packet-switched network, completion status data indicating that preparation of the target item identifiers has been completed, and updates a status record in the storage device in association with the terminal device.

12. The system according to claim 1, wherein the circuitry further generates delivery instruction data including a delivery order and a movement route by executing a route optimization operation on the basis of location data associated with the terminal device and preparation completion data received from the second device, and transmits the delivery instruction data to a third device via the packet-switched network.

13. The system according to claim 12, wherein the route optimization operation computes the movement route by applying a shortest-path or minimum-cost algorithm to a graph representing delivery locations, and the delivery instruction data further includes an estimated arrival time computed from the movement route and a current position of the third device.

14. The system according to claim 1, wherein the circuitry receives, from the terminal device via the packet-switched network, evaluation data including a satisfaction score and a free-text evaluation associated with the structured output data, and stores the evaluation data in the storage device in association with the structured output data and the constraint parameters.

15. The system according to claim 14, wherein the circuitry applies a machine learning algorithm to the evaluation data and the historical preference data to update preference weight parameters stored in the storage device, and incorporates the updated preference weight parameters into subsequent prompt data structures to bias the generative neural network model toward higher-rated output patterns.

16. The system according to claim 1, wherein the circuitry determines, for each item in the component data, conformity to nutritional condition data and restricted-component condition data stored in the storage device, and excludes or substitutes items that do not conform before generating the instruction data.

17. The system according to claim 16, wherein the circuitry generates authentication data associated with a pickup event, transmits the authentication data to the terminal device, and stores the authentication data in the storage device in association with a storage apparatus identifier, the authentication data being required for release of a prepared item from the storage apparatus.

18. A system comprising:
circuitry configured to:
receive, via a communication interface coupled to a packet-switched network, preference data and feedback data from a terminal device, and retrieve historical preference data associated with the terminal device from a storage device;
apply a natural language processing operation to the preference data to extract constraint tokens classified into restriction, affinity, and quantity categories, and combine the constraint tokens with the historical preference data to generate constraint parameters;
construct a prompt data structure including the constraint parameters and an instruction sequence directing a generative neural network model comprising a transformer architecture with a token embedding layer, a positional encoding mechanism, a plurality of self-attention layers, and feedforward sublayers to generate structured output data in a predetermined output format, the structured output data comprising procedure data and component data;
evaluate the structured output data against the constraint parameters to verify compliance of the component data, and when a non-compliant item is detected, construct a revised prompt data structure requesting substitution;
generate instruction data including operation parameters derived from the procedure data and target item identifiers derived from the component data, and transmit the instruction data to a second device at a preparation facility via the packet-switched network;
generate delivery instruction data including a movement route computed by a route optimization operation on the basis of location data associated with the terminal device, and transmit the delivery instruction data to a third device via the packet-switched network; and
receive evaluation data from the terminal device, apply a machine learning algorithm to the evaluation data and the historical preference data to update preference weight parameters stored in the storage device for incorporation into subsequent prompt data structures.

19. The system according to claim 18, wherein the circuitry generates authentication data associated with a pickup event, transmits the authentication data to the terminal device, stores the authentication data in the storage device in association with a storage apparatus identifier, and determines conformity of each item in the component data to nutritional condition data and restricted-component condition data before generating the instruction data.

20. A method performed by circuitry of a server coupled to a packet-switched network via a communication interface, the method comprising:
receiving, via the communication interface, preference data and feedback data from a terminal device;
retrieving, from a storage device, historical preference data associated with the terminal device;
analyzing the preference data, the feedback data, and the historical preference data to generate constraint parameters, and constructing a prompt data structure including the constraint parameters and an instruction sequence directing a generative neural network model to generate structured output data conforming to the constraint parameters in a predetermined output format;
transmitting the prompt data structure to the generative neural network model and receiving response data including the structured output data comprising procedure data and component data; and
generating instruction data on the basis of the structured output data and transmitting the instruction data to a second device via the packet-switched network.
